(19) United States
(12) Patent Application Publication
(10) Pub. No.: US 2024/0191251 A1
(43) Pub. Date: Jun. 13, 2024

(54) RECOMBINANT HERPESVIRALES VECTOR

(71) Applicant: Solid Biosciences Inc., Charlestown, MA (US)

(72) Inventors: Peter Pechan, Newton, MA (US); Sarath Chandra Mandava, Malden, MA (US)

(21) Appl. No.: 18/285,630

(22) PCT Filed: Apr. 5, 2022
(86) PCT No.: PCT/US2022/023545

§ 371 Date: Oct. 4, 2023

Related U.S. Application Data
(60) Provisional application No. 63/285,338, filed on Dec. 2, 2021.
(60) Provisional application No. 63/170,749, filed on Apr. 5, 2021.

(51) Int. Cl. C12N 15/86; C07K 14/47

(52) U.S. Cl. CPC C12N 15/86; C07K 14/4716; C12N 2710/16643; C12N 2710/16652; C12N 2750/14122; C12N 2750/14144; C12N 2830/50

(57) ABSTRACT

The invention described herein provides a recombinant replication-defective virus derived from Herpesvirales order, comprising a defective ICP27 gene that impairs or otherwise does not support replication of the HSV, or a functional equivalent gene thereof; a gene-of-interest (GOI) flanked by AAV ITR sequences inserted at, within, or replacing a non-essential or replaceable essential locus of the HSV; and a coding sequence for AAV Rep and Cap proteins inserted at, within or replacing the non-essential or replaceable essential locus of the HSV. The invention also provides production cell lines for such recombinant replication-defective virus, wherein the cell lines have a coding sequence for ICP27 or a functional equivalent thereof, and wherein the coding sequence has no or minimal sequence overlap with the virus characterized by the defective ICP27 gene that impairs or otherwise does not support replication of the HSV. Methods of using such recombinant replication-defective virus and production cell lines are also provided. rAAV produced using such recombinant replication-defective virus and/or production cell lines and methods of making such rAAV are also provided.

### REFERENCE TO RELATED APPLICATIONS

This application claims priority to and the benefit of the filing dates of U.S. Provisional Patent Application No. 63/170,749, filed on Apr. 5, 2021, and 63/285,338, filed on Dec. 2, 2021, the entire contents of each of the above referenced applications, including any drawings and sequence listings, are incorporated herein by reference.

### BACKGROUND OF THE INVENTION

Recombinant adeno-associated viral (rAAV) vectors production using co-infection with recombinant herpes simplex virus type 1 (rHSV) vectors is a very efficient method of generating a large amount of rAAV particles (Conway et al., Gene Therapy 6:986-993, 1999; Booth et al., Gene Therapy 11:829-837, 2004; Adamson-Small et al., Mol. Ther. Med. Clin. Dev. 3:16031, 2016). The production method relies on the role played by the HSV in AAV life cycle as a helper virus for replication in permissive cells. Therefore, the rHSV virus can serve both as a helper and as a shuttle to deliver the necessary AAV functions that support AAV genome replication and packaging to the producing cells.

Some rHSV vectors for this system are engineered from a replication-deficient variant of HSV type 1 virus d27-1 with a 1.6-kb deletion in a gene encoding the viral Infected Cell Protein 27, or ICP27 (Rice and Knipe, J. Virol. 64:1704-1715, 1990). ICP27 is a 512-amino acid protein, also known by names based on its molecular weight, as Immediate Early 63 (IE63) or Viral Molecular Weight 63 (Vmw63) protein, or by its location on HSV-1 genome as Unique Long 54 (UL54) gene. ICP27 is one of the first proteins to be expressed in cells infected with HSV-1 and is absolutely essential for viral replication in cell culture. In the absence of ICP27, the rHSV genome cannot replicate, unless ICP27 is provided in trans, for example, by V27 cells, a Vero cell derivative stably transformed with 2.45-kb BamHI-HpaI fragment containing UL54 and part of UL55 genes to express ICP27 (Rice and Knipe, J. Virol. 64:1704-1715, 1990). Other reported ICP27-complementing cell lines were Vero-derived 2-2 cells and BHK21-derived B130 cells, both stably transformed with 2.42-kb BamHI-SstI fragment also containing UL54 and part of UL55 genes (Smith et al. Virology 186: 74-86, 1992; Howard et al. Gene Therapy 5:1137-1147, 1998).

V27 cell line can be used for large-scale manufacturing of rHSV stocks used for rAAV production (Penaud-Budloo et al., Mol. Ther.: Med. & Clin. Dev. 8:166-180, 2018). rHSV stocks can be prepared by infecting monolayers of V27 cells in flasks, or alternatively, in suspension using micro-carriers. The resulting rHSV stocks are generally harvested after 3-4 days, and concentrated (Knop and Harell, Biotechnol. Prog. 23:715-721, 2007; Adamson-Small et al., Mol. Ther. Med. Clin. Dev. 3:16031, 2016).

However, when a replication-defective virus vector is propagated in a helper cell line, there is a high probability that the final stock will contain a sub-population of virus that has obtained replication competency through the process of homologous recombination (HR) between the viral genome and the integrated viral gene(s) present in the cellular genome observed also during production of adenovirus (Ad) vectors, where homologous recombination between E1 gene-deleted Ad vectors genomes and the integrated E1 sequences in 293 cells frequently resulted in replication-competent Ads (RCA) that contaminated stocks of E1-vectors (Hehir et al., Journal of Virology. 70:8459-8467, 1996). It is no exception that the manufacture process using the current d27-1 recombinant HSV (rHSV) vector in V27 cells can also lead to the generation of a “wild-type like” replication-competent HSV (rcHSV) contamination of the rHSV lots.

Specifically, replication-competent HSV (rcHSV), or ICP27 revertants, may arise during amplification of rHSV in V27 by homologous recombination (Ye et al., Hum. Gene Ther. Clin. Dev. 25:212-217, 2014). Low levels of rcHSV have been shown in rHSV stocks, reported less than 1 PFU per 3×10⁸ rHSV PFUs (Penaud-Budloo et al., Mol. Ther.: Med. & Clin. Dev. 8:166-180, 2018).

However, any rcHSV virus which would behave phenotypically as wild-type virus, and especially in case in vectors which have disrupted thymidine kinase (TK) gene (Conway et al., Gene Therapy 6:986-993, 1999), may negatively impact the ability of using a common anti-HSV drug like acyclovir or ganciclovir during potential infection. This would pose a serious problem to the therapeutic use of replication-defective stocks, because HSV-1 has the potential for uncontrolled viral replication and has a potential to cause a crippling form of encephalitis as a result of viral spread in the brain (Asenbauer et al., Neuropediatrics 29:120-123, 1998; Gurses et al., Annals of Tropical Paediatrics. 16:173-175, 1996; Yamada et al. Journal of Neurology, Neurosurgery & Psychiatry 74:262-264, 2003).

Thus, there is a need to improve rHSV production processes, and in particular, known processes that utilize the d27-1 rHSV vector and V27, Vero 2-2 or BHK B103 cells.

### SUMMARY OF THE INVENTION

The vectors described herein provides new rHSV vectors that not only have a larger, relatively complete (e.g., complete) ICP27 gene deletion in its backbone, compared to the existing d27-1 ICP27 deletion, but also comprises a gene of interest (GOI) flanked by AAV ITR sequences, and AAV Rep and Cap protein coding sequences, all inserted at, into, or within a locus (e.g., a non-essential locus) of HSV, such as the UL43 locus, the UL54 locus, or in other genes. The locus may be a replaceable essential locus. In certain embodiments, the replaceable essential locus may be the UL54 locus. In certain embodiments, the TK locus is not considered such non-essential locus (i.e., inserted at, into, or within a non-essential locus that is not the TK locus).

One advantage of such HSV based vectors is that they have been shown to obviate contamination of rcHSV generated during the production of rHSV using the d27-1-based complementation system in a rHSV production cell line. Another advantage of such HSV based vectors is that they enable rAAV production by a single infection of HSV/AAV hybrid vector, but are replication-deficient with preferably, a complete deletion in the ICP27 gene. Further, the HSV vectors of the invention carry both the GOI (to be packaged into AAV viral particles), and the rep and cap cassettes required for AAV packaging in the same locus (i.e., in ULA3, UL54, or in other genes). Hence such vectors are sometimes referred to herein as the so-called all-in-one (or A-I-O or AIO) vector, compared to similar systems in which the GOI and the rep/cap coding sequences are carried by separate HSV vectors.

Surprisingly, compared to the HSV vectors carrying only the GOI but not the rep/cap coding sequences tandemly inserted in/at/within one locus (such as UL43 or UL54), it was found that such A-I-O HSV vectors produced significantly less mis-packaged AAV viral particles encompassing vector sequences outside the flanking ITR sequences.

Further surprisingly, A-I-O HSV vectors of the present invention have produced a high yield of AAV viral particles also in an ICP27-expressing HSV packaging cell line (e.g., V75, sBHK-27), comparable to a rAAV production in non-complementing cells (e.g., HEK293 Expi293F), thus providing a potential single-vessel production of both HSV and rAAV, without a need of producing HSV separately, its processing (concentrating by TFF) tittering, and viral banking.

It is also found that the AAV viral stocks produced by the A-I-O vector of the invention, either in suspension (e.g., sBHK27) cells or adherent (e.g., V75) cells, have shown very high expression level that is equivalent to that of the AAV viral stocks produced by the conventional triple transfection method.

Thus the invention described herein provides a recombinant replication-defective Herpes Simplex Virus (HSV) vector, comprising: (1) a defective ICP27 gene that impairs or otherwise does not support replication of said HSV; (2) a gene of interest (GOI) flanked by AAV ITR sequences, inserted at or within a non-essential HSV locus (e.g., the UL43 locus) or a replaceable essential locus (e.g., the UL54 locus), or any other non-essential loci (optionally, such other non-essential locus is not the TK locus), of the HSV and, (3) an expression cassette comprising a coding sequence for AAV Rep and Cap proteins, inserted at or within the non-essential locus (such as the UL43 locus) or a replaceable essential locus (such as the UL54 locus), or any other non-essential loci (optionally, such other non-essential locus is not the TK locus), of the HSV.

In certain embodiments, both the GOI in (2) (sometimes referred to as the “pro-AAV cassette”) and the RepCap coding sequence in (3) (sometimes referred to the “RepCap cassette”) are inserted in tandem with or without intervening vector sequence (such as interveneing BACmid or plasmid sequence) in between.

In certain embodiments, both the GOI in (2) (sometimes referred to as the “pro-AAV cassette”) and the RepCap coding sequence in (3) (sometimes referred to the “RepCap cassette”) are inserted in tandem without intervening vector sequence (such as interveneing BACmid or plasmid sequence) in between.

In certain embodiments, the GOI in (2) is inserted more distal to the Us region in relation to the RepCap coding sequence in (3).

In certain embodiments, the GOI in (2) is inserted more proximal to the Us region in relation to the RepCap coding sequence in (3).

In certain embodiments, the defective ICP27 gene comprises/is a complete deletion of the UL54/ICP27 locus of the HSV (e.g., a complete deletion of SEQ ID NO: 2, or a coding sequence for SEQ ID NO: 1).

In certain embodiments, the rHSV vector comprises an intact UL42 locus and an intact UL44 locus and/or an intact UL53 locus and an intact UL55 locus, optionally further comprising an intact UL23 locus and/or an intact US5 locus.

In certain embodiments, the HSV is Herpes Simplex Virus-1 (HSV-1) or Herpes Simplex Virus-2 (HSV-2).

In certain embodiments, the HSV is a strain of HSV-1, such as KOS, KOS 1.1, KOS 1.1A, KOS63, KOS79, McKrae, Stain 17, F17, McIntyre, or viruses resulting from reconstitution of HSV BAC E. coli DNAs, such as BAC HSV KOS-37 or BAC HSV 17-37 (Gierasch et al., J Virol Meth 135:197-206, 2006) and other BAC HSV DNAs.

In certain embodiments, the HSV is HSV37.

In certain embodiments, the GOI is a functional equivalent of dystrophin (e.g., a dystrophin minigene encoding a functional micro-dystrophin protein).

In certain embodiments, the GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17. R23, and R24 spectrin-like repeats of the full-length dystrophin protein (such as the microdystrophin gene described in PCT/US2016/013733).

In certain embodiments, the AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, or AAV-DJ.

In certain embodiments, the tropism of the AAV include skeletal muscle (such as AAV1, AAV6, AAV7, AAV8, or AAV9, preferably AAV9).

In certain embodiments, the AAV ITR, the AAV Rep, and the AAV Cap are from the same or different AAVs.

In certain embodiments, the AAV ITR is AAV2 ITR, the AAV Rep is Rep2 from AAV2, and the AAV Cap is Cap9 from AAV9.

In certain embodiments, the coding sequence for AAV Rep and Cap proteins is under the transcriptional control of a promoter, such as an AAV p5 promoter, an upstream HSV promoter, a modified p5 promoter lacking RBE (Rep-Binding Element), or a ubiquitous promoter (such as CMV promoter, EF1a promoter, CAG promoter, CB promoter etc).

In certain embodiments, the rHSV vector further comprises a removable transcription unit inserted at or within the UL43 locus of the HSV, wherein the removable transcription unit comprises a selection marker (such as an antibiotic resistance gene, including but not limited to kanamycin resistance gene Kan^{R} or zeocin resistance gene Zeo^{R}) under the transcriptional control of a selection marker promoter (such as a Kan^{R} or Zeo^{R} promoter).

In certain embodiments, the rHSV vector further comprises a removable transcription unit inserted at or within the UL54 locus of the HSV, wherein the removable transcription unit comprises a selection marker (such as an antibiotic resistance gene, including but not limited to kanamycin resistance gene Kan^{R} or zeocin resistance gene Zeo^{R}) under the transcriptional control of a selection marker promoter (such as a Kan^{R} or Zeo^{R} promoter).

In certain embodiments, the removable transcription unit is flanked by a pair of flippase recognition target (FRT) sites.

In certain embodiments, the GOI encodes a CRISPR/Cas effector enzyme.

In certain embodiments, the CRISPR/Cas effector enzyme is a Class 2, Type II, V, or VI effector enzyme, such as a Cas9 or a variant thereof.

In certain embodiments, CRISPR/Cas effector enzyme lacks endonuclease activity (dCas, such as dCas9). In certain embodiments, the Cas or dCas is further fused to a base editor, such as a cytosine base editor (CBE, e.g., APOBEC, BE1, BE2, BE3, Target-AID base editor, SaBE3, BE3 PAM variants, BE3 editing window variants, AID, CDA1, APOBEC3G, HF-BE3, BE4, BE4max, and AncBE4max), an adenine base editor (ABE, e.g., ABE7.10, ABE 6.3, ABE7.8, ABE7.9, ABEmax, ABE8c(TadA-8c V106W), ABE8 and variants thereof), or a dual base editor (SPACE, A&C-BEmax).

In certain embodiments, the GOI encodes a therapeutic antibody or an antigen-binding fragment thereof, or a vaccine epitope from a virus, a bacterium, or a cancer-specific antigen.

Another aspect of the invention provides a recombinant replication-defective Herpes Simplex Virus (HSV) viral particle, comprising the rHSV vector of the invention.

Another aspect of the invention provides a host cell comprising the recombinant rHSV vector of the invention.

In certain embodiments, the host cell does not comprise a coding sequence for ICP27, and the host cell optionally further comprises helper virus proteins required for AAV packaging.

In certain embodiments, the host cell is a HEK293 cell (such as an Expi293F cell, or an Expi293 cell engineered to express the ICP27 gene), a HeLa cell, an A549 cell, a BHK cell (such as sBHK27), or an insect cell (such as Sf9).

In certain embodiments, the host cell comprises a coding sequence for, and is capable of expressing ICP27 or a functional equivalent thereof (such as V75 cells or Expi293 cells).

In certain embodiments, the host cell comprises: (1) a coding sequence for said ICP27 or said functional equivalent thereof, operatively linked to a promoter capable of directing the transcription of said coding sequence in the host cell; (2) a polyadenylation site 3′ to the coding sequence; and, (3) optionally, one or more multi-cloning site(s).

In certain embodiments, the ICP27 has the amino acid sequence of SEQ ID NO: 1, or is at least 60%, 70%, 75%, 80%, 85%, 90%, 95%, 97%, 98%, 99%, 99.2%, 99.4%, 99.6%, or 99.8% identical to SEQ ID NO: 1.

In certain embodiments, the promoter comprises nucleotides 1-538 of SEQ ID NO: 2, nucleotides 127-538 of SEQ ID NO: 2, nucleotides 113,139-113,550 of GenBank Accession No. KT887224, or nucleotides 113,013-113,550 of GenBank Accession No. KT887224.

In certain embodiments, the coding sequence is partially or fully codon-optimized for expression in a mammalian host cell.

In certain embodiments, the most 3′ 300-350 nucleotides of the coding sequence are codon-optimized for expression in the mammalian host cell.

In certain embodiments, the polyadenylation site is a bovine growth hormone (bGH) polyadenylation site.

In certain embodiments, the coding sequence for said ICP27 comprises a mutation that reduces inhibition of host cell pre-mRNA splicing, while permitting HSV late gene expression.

In certain embodiments, the mutation is vBS3.3 double mutation, vBS4.3 double mutation, or vBS5.3 double mutation.

In certain embodiments, the host cell is a Vero cell (such as a V75 cell) or a BHK cell (such as a sBHK27 cell).

Another aspect of the invention provides a method of simultaneously propagating/amplifying/producing the recombinant replication-defective HSV viral particle of the invention, and a recombinant adeno-associated virus (AAV) viral particle encapsidating the GOI flanked by AAV ITR, the method comprising: infecting the host cell of the invention with the recombinant replication-defective HSV viral particle of the invention.

In certain embodiments, the method further comprises harvesting the recombinant replication-defective HSV of the invention from the infected host cell of the invention.

Another aspect of the present invention includes an HSV vector comprising a gene of interest (GOI) flanked by AAV ITR sequences, inserted at or within a non-essential locus (e.g., the UL43 locus) or a replaceable essential locus (e.g., the UL54 locus) of the HSV and an expression cassette comprising a coding sequence for AAV Rep and Cap proteins, inserted at or within the non-essential locus (e.g., the UL43 locus) or the replaceable essential locus (e.g., the UL54 locus) of the HSV.

Another aspect of the invention provides a method of producing a recombinant Adeno-Associated Virus (rAAV) comprising a gene of interest (GOI) coding sequence flanked by AAV ITR sequences, said method comprising infecting a production host cell of the invention, with the recombinant replication-defective HSV viral particle of the invention.

In certain embodiments, the production cell line is BHK, Vero, or HEK293.

Another aspect of the invention provides a method to improve the quality, potency, expression level, and/or purity of the AAV viral particles or viral stocks produced by a dual HSV vector system, in which one HSV vector comprises coding sequence for a GOI flanked by a pair of AAV ITR sequences, and another HSV vector comprises coding sequences for the AAV Rep and Cap proteins, the method comprising infecting a suitable AAV production cell line with an A-I-O HSV vector of the invention comprising both the coding sequence for the GOI flanked by the AAV ITR sequences, and the coding sequences for the AAV Rep and Cap proteins (e.g., both inserted into the same locus of the A-I-O HSV vector, such as the UL43 locus).

It should be understood that any one embodiment of the invention, including those only described in the Examples, claims, or one of the subsections, can be combined with any other one or more embodiments, unless improper or expressly disclaimed.

### BRIEF DESCRIPTION OF THE DRAWINGS

FIG. 1A is a schematic (not to scale) drawing showing one exemplary embodiment of the A-I-O HSV vector of the invention. The ICP27/UL54 locus is deleted (A), while the TK/UL23 locus is intact (TK+). At the UL43 locus, there is an insertion or replacement comprising a GOI flanked by AAV ITR sequences, as well as the AAV Rep and AAV Cap coding sequences. The adjacent intact upstream UL36, UL37, and UL42 loci and intact downstream UL44 locus are also shown. The black bars represent the HSV Unique Long (UL) and Unique Short (US) sequences.

FIG. 1B is a more detailed schematic drawing showing the different sequence elements in FIG. 1A, including the 5′ ITR, the promoter, the GOI, and the 3′ ITR in the pro-AAV cassette; the p5 promoter-driven rep and cap coding sequence in the RepCap expression cassette; the optional Zeocin resistance selection marker gene Zeo^{R} under the transcriptional control of its own promoter (pZ), flanked by the frt recognition sequences for FLP-mediated removal; as well as the intact UL42 locus and UL44 locus, and the remnants of the UL43 locus. The deleted ICP27/UL54 locus is not shown. Note that the FRT sites here are an optional design feature of the illustrated embodiment. In addition to zeocin, any other bacterial resistant markers, such as kanamycin, can also be used. The box between the pro-AAV cassette and the RepCap cassette represents remnant (about 200 bp) vector plasmid sequence that can be deleted (not shown in FIG. 1A).

FIG. 1C is an even more detailed version of an exemplary A-I-O HSV vector of the invention shown in FIGS. 1A and 1B, including the various restriction enzyme sites; a microdystrophin gene (MD5) driven by the muscle-specific CK8 promoter and including a polyA signal sequence AAUAAA; the AAV2 Rep and AAV0 Cap coding sequences including polyA signal AAUAAA; and the pEM7 promoter-driven Zeo^{R}.

FIG. 1D is a schematic (not to scale) drawing showing another exemplary embodiment of the A-I-O HSV vector of the invention. The ICP27/UL54 locus is deleted (Δ), while the TK/UL23 locus is intact (TK+). At the same UL54 locus, there is an insertion or replacement comprising a GOI flanked by AAV ITR sequences, as well as the AAV Rep and AAV Cap coding sequences. The adjacent intact upstream UL53 locus and the intact downstream UL55 locus are also shown. The black bars represent the HSV Unique Long (UL) and Unique Short (US) sequences. The box between the pro-AAV cassette and the RepCap cassette in FIG. 1A, representing any remnant (about 200 bp) vector plasmid sequence, is deleted and not shown here.

FIG. 1E is a more detailed schematic drawing showing the different sequence elements in FIG. 1D, including the 5′ ITR, the promoter, the GOI, and the 3′ ITR in the pro-AAV cassette; the p5 promoter-driven rep and cap coding sequence in the RepCap expression cassette; the optional Kananmycin resistance selection marker gene Kan^{R} under the transcriptional control of its own promoter (pZ), and, different from the design in FIG. 1B, lacks the optional frt recognition sequences for FLP-mediated removal; as well as the intact UL53 locus and UL55 locus. Note that the optional design feature of FRT sites are not included in this specific embodiment, and also note that any other bacterial resistant markers, not just kanamycin resistance gene, can also be used. There is no box between the pro-AAV cassette and the RepCap cassette in this exemplary construct—the remnant (about 200 bp) vector plasmid sequence is deleted.

FIG. 2A is a schematic drawing showing simultaneous production of HSV and AAV in ICP-27-expressing HSV packaging cell line V75, and AAV production in ICP-27-negative AAV production cell line Expi293F, using A-I-O HSV vectors (e.g., d43d27 HSVgoi-repcap) of the present invention.

FIG. 2B is an alternative schematic drawing showing (2.) simultaneous production of HSV and AAV in ICP-27-expressing HSV packaging cell line V75, and (1.) AAV production in ICP-27-negative AAV production cell line 293, using A-I-O HSV vectors (e.g., d43d27 HSVgoi-repcap) of the present invention.

FIG. 3A is a high-level illustration of medium scale production of AAV viral particles using A-I-O (e.g., d43d27 HSV) vectors of the present invention in ICP27-expressing cell lines (such as sBHK27 cells for suspension growth, or V75 cells for adherent culture). Briefly, for example, V75 cells are initially grown in a T150 tissue culture flask and produced passage P2 of the A-I-O HSV virus encompassing a microdystrophin (μDys) coding sequence as the GOI, with the plaque forming units (pfu) titer of 1.4×10⁶ (1.4E6) pfu/mL. The ddPCR copy titer of the μDys-carrying HSV and subsequently produced AAV in the lysate reaches 1E10 vg/mL, while the UL36 and UL42 sequence copies have a titer of about 2.63E8 vg/mL 3.81E8 vg/mL (or about 3% or ˜4% of the μDys copies). Passage P2 of the A-I-O HSV (10 mL or 1.4E7 pfu of each) was used to infect 1E9 of suspension sBHK27 cells (A153) in 450 mL of serum-free media (SFM) in a tissue culture shake flask (MOI of 0.014); and 2E8 of V75 cells growing as adherent culture in tissue culture cell stack adherent V75 cells (MOI of 0.07). After 7 days post infection (7 dpi), the AAV viral particles are harvested by benzonase & triton lysis, HDF, and FPLC.

FIG. 3B shows the relative potency of AAV Lots produced from an All-In-One (A-I-O) HSV vector systems of the present invention—A-I-O AAV #153 was produced in suspension sBHK27 cells, and A-I-O AAV #154 was produced in adherent V75 cells. The various lots of AAV viral particles were assessed for their respective potency in AAV-infected mouse differentiated myoblast C2C12 cells assay. AAV-Dual #1 and AAV-Dual #2 samples were both produced by infection with dual HSV system in suspension 293 cells coinfected with both HSV-goi and HSVrepcap. AAV-Dual #2 was used as the reference (100%) to obtain the relative potency of the other AAV samples in the assay. Reference standard material for in vitro expression assays, e.g., PB19017, is considered a high potency PD lot. A typical GMP lot produced by a dual HSV vector system tends to have a potency between 12-30% of that of the reference standard material, e.g., a PB19017 lot. All-In-One (A-I-O) HSV produced AAV samples, A-I-O AAV #153 and A-I-O AAV #154 were found to have 23-25 fold- or 15-16 fold-higher microdystrophin expression than dual HSV system produced AAV LotsAAV-Dual #1 or AAV-Dual #2, respectively.

FIG. 4 shows the yield of AAV viral particles produced by suspension culture of sBHK27 cells, at various stages of purification until after FPLC. As a comparison, mis-packaged AAV viral particles encompassing UL42 sequences are titered at 5.51E9 of total vg, representing 0.35% of the final titer of the u Dys-containing AAV (1.58E12 vg), while UL36 sequences, which are most likely not mispackaged and represent just a contamination of the final stock (e.g., residual DNA after purification), were at a total of 3.02E7 vg (or about 0.0019%) of the final u Dys titer (a 184-fold drop).

FIG. 5 shows the yield of AAV viral particles produced by adherent culture of V75 cells, at various stages of purification until after FPLC. As a comparison, mis-packaged AAV viral particles encompassing UL42 sequences are titered at 1.47E10 of total vg, representing 0.60% of the final titer of the u Dys-containing AAV (2.44E12 vg), while UL36 sequences, which are most likely not mispackaged and represent just a contamination of the final stock, were at a total of 2.61E8 vg, or about 0.011% of the final u Dys titer (a 55-fold drop).

FIG. 6 shows an illustrative workflow for continuous HSV and AAV production in one vessel in ICP27-expressing sBHK27 cells, using A-I-O HSV vectors of the present invention.

FIG. 7 shows the dual HSV vectors with UL43 insertion for AAV production (d43RC9&GOI). Co-infection of a suitable production cell line (such as HEK293 cells) with both HSV vectors in the presence of AAV packaging helper can be used to produce AAV viral particles encompassing the GOI in the HSV vectors. However, a certain percentage of mis-packaged AAV viral particles, encompassing HSV vector sequences outside the AAV ITR sequences, was also produced. See FIG. 8.

FIG. 8 shows significantly reduced AAV viral particles with mis-packaged HSV DNA when produced in Expi293 cells using the subject A-I-O HSV vectors. The left panel shows that in the dual HSV vector system, up to about 20% of AAVs have mis-packaged UL42 sequences when the GOI was inserted at the UL43 locus. The right panel shows that in seven A-I-O vectors tested, up to 1.5% (at least about 14-fold reduction) mis-packaged AAV viral particles in 4 of the 7 vectors tested, and undetectable mis-packaged AAV viral particles in the remaining 3 tested A-I-O HSV vectors.

FIG. 9 shows, with respect to the present invention, consistent over-production of AAV viral particles in both ICP-27-expressing V75 cells infected by A-I-O HSV vectors, and AAV production in ICP-27 negative Expi293F cells infected by the same A-I-O HSV vectors.

FIG. 10 shows titers (in vg/mL) for a micro-dystrophin (μD) coding sequence as the GOI, and HSV UL53 mispackaged into AAV viral particles, produced by infecting sBHK27 cells at both low (0.02) and high (0.1) MOI (Multiplicity of Infection), produced using A-I-O HSV vectors of the present invention, with the insertion at the UL54 locus (AIO-U154 HSV).

FIG. 11 shows a low percentage of mispackaging in microdystrophin (μD)-encoding AAV viral particles, as measured by mispackaged HSV DNA at the UL53 locus. The AAV viral particles were produced by infecting sBHK27 cells at both low (0.02) and high (0.1) MOI, with A-I-O HSV vectors of the present invention with the insertion at the UL54 locus (AIO-U154 HSV).

### DETAILED DESCRIPTION OF THE INVENTION

### 1. Overview

Conventional HSV-based AAV production system utilizes two HSV vectors (the dual HSV vector system), one with a gene of interest (GOI) flanked by AAV ITR sequences, for packaging into the AAV viral particles; the other with an expression cassette encoding the AAV Rep and Cap proteins required for AAV packaging. These HSV vectors are typically also replication defective with respect to the HSV, due to, for example, the lack of a functional ICP27 gene at the UL54 locus, which is required for HSV replication. Thus, HSV viral stocks can be produced in a suitable HSV producing cell line that supplies the required ICP27 protein. The separately produced dual HSV vectors (the GOI HSV and the rep/cap HSV) are then used together to co-infect an AAV producing cell line to provide the Rep and Cap proteins, as well as any other required helper functions for AAV packaging, which may be supplied by the HSV vector, the AAV production cell line, and/or a co-infected helper virus (such as adenovirus).

The d27-1 rHSV-V27-based vector-host cell system is one such conventional dual HSV-based AAV production system. The d27-1 rHSV vector, either with the AAV rep/cap coding sequence or with the AAV ITR-flanked GOI, has a nearly complete (but incomplete) ICP27/UL54 locus deletion, and the V27 HSV producing cell line has a portion of the HSV genome encoding the ICP27 protein.

One drawback of the d27-1 rHSV-V27 system is that there is an 815-nucleotide overlap between the sequences in the d27-1 virus and the HSV-1 sequence integrated into the complementing V27 cells. Similar or larger size of the overlap also exists within the other ICP27-deleted viruses and similar complementing cells, i.e., 2-2 cells or B130 cells. These sequence overlap enables the occasional homologous recombination between the sequences in ICP27-deleted viruses and the HSV-1 sequences integrated in ICP27 complementing cells, resulting in the appearance of wild type-like replication-competent contaminating viruses (rcHSV) in the ICP27-deleted virus stocks. These stocks then grow also on non-complementing cells where propagation of ICP27-deleted viruses should be restricted. This has been observed by increased virus stocks cytotoxicity and generation of viral plaques on non-complementing cells. The presence of the rcHSV in an AAV viral stock for gene therapy can pose a high and usually unacceptable risk for gene therapy patients, and may not satisfy the regulatory approval standard for the AAV gene therapy products. These undesirable affects are now alleviated by the rHSV vectors and methods of the invention, by, preferably, completely removing the regions creating these overlaps.

Another drawback of such traditional dual HSV vector system is the relatively high labor and costs associated with operating the dual HSV vector system. The system requires separate production of two HSV vectors, accurate titering of two HSV vector lots (i.e., the HSV vector with the GOI construct, and the HSV vector encoding the rep/cap expression cassette). Further, tuning the MOIs of these two HSV viral stocks for infection while producing AAV is required. These operational disadvantages are significantly alleviated if not completely eliminated by the A-I-O vector systems of the present invention, where operational cost is cut in half—thus enabling higher overall production, a common rate-limiting factor for AAV-based gene therapy, especially for those requiring repeated therapy and/or systemic delivery.

The recombinant HSV vectors of the invention described herein improves the conventional system by inserting both the GOI constructs and the rep-cap expression cassette into a single non-essential locus of the HSV (such as the UL43 locus), or a replaceable essential locus of the HSV (such as the UL54 locus). Thus, AAV production using the All-In-One (A-I-O) HSV vectors of the present invention can be based on an HSV vector containing both the AAV ITR-flanked GOI (e.g., ITR-GOI-ITR) and the rep/cap expression cassette.

Surprisingly, it was found that the A-I-O vectors of the present invention produced much higher titer of AAV in an ICP-27-expressing HSV producing cell line (such as the adherent V75 cell line or the suspension sBHK27 cell line described herein).

Further surprisingly, it was found that the A-I-O vectors of the present invention largely eliminated/significantly reduced undesirable AAV mis-packaging, for example, when the insertions are at the UL43 locus or the UL54 locus, compared to corresponding conventional dual HSV vector system. Specifically, in such corresponding conventional dual HSV vector systems, up to about 20% of the produced AAV viral particles may have HSV vector sequences unrelated to the GOI flanked by the AAV ITR sequences. The rate of mis-packaging is dramatically reduced by at least 10-fold to 100-fold at the UL43 or UL54 locus using the A-I-O vector of the invention.

Thus, the A-I-O vectors of the invention may provide not only reduced mispackaging and improve product quality, but also potentially higher AAV viral particle yield.

Propagation of the new replication-defective virus of the present invention (e.g., rHSV-1) preferably harboring a large ICP27 deletion (e.g., a complete deletion of the ICP27-encoding UL54 gene), or which impairs or otherwise does not support replication of the HSV, together with all the required elements for producing AAV viral particles encapsidating a GOI, either, e.g., in an adherent Vero cell (such as V75) or serum-free suspension adapted BHK cell line (such as sBHK27), will preferably have no overlap between the ICP27 gene integrated in the cellular genome and viral genome of the rHSV with complete UL54 gene deletion, and will thus enable the production of rHSV stocks free of the replication-competent rcHSV virus, with a drastically reduced rate of mis-packaged AAV viral particles.

The rcHSV-free A-I-O rHSV stock of the invention can be very useful for large scale production of rAAV, which in turn can be used in gene therapy, for expression of therapeutic proteins (peptides, enzymes, antibodies, etc.), oligonucleotides (i.e., shRNAs, miRNAs), and gene editing and silencing tools (CRISPR-Cas, TALEN, shRNA, miRNA and others), etc.

Thus one aspect of the invention provides a recombinant replication-defective Herpes Simplex Virus (HSV) vector, comprising: (1) a defective ICP27 gene that impairs or otherwise does not support replication of said HSV; (2) a gene of interest (GOI) flanked by AAV ITR sequences, inserted, e.g., at or within the UL43 or UL54 locus of the HSV, or any other locus of the HSV; and, (3) an expression cassette comprising a coding sequence for AAV Rep and Cap proteins, inserted at or within the same (e.g., the UL43 or UL54) locus of the HSV. Optionally, said any other locus is a non-essential locus (such as the TK locus, or a non-essential locus that is not the TK locus); or an essential locus (such as UL54), in which case the essential function of the locus can be provided by a host cell when replication of the HSV vector is desired, which may also be understood to be a replaceable essential locus.

In certain embodiments, the ratio between (1) the AAV viral particles having a mispackaged HSV vector sequence (e.g., a mispackaged HSV vector sequence adjacent to the insertion locus—for example, a sequence from the UL42 or the UL53 locus, when the Pro-AAV and RepCap cassettes are both inserted in the UL43 or UK54 locus, respectively) and (2) the AAV viral particles having the correctly packaged GOI sequence, is no more than about 1.5%, 1%, 0.5%, 0.4%, 0.3%, 0.2%, 0.1%, 0.09%, 0.08%, 0.07%, 0.06%, 0.05%, 0.04%, 0.03%, 0.02%, 0.01% or less; or is between about 0.01 to about 2%, about 0.05 to about 2%, about 0.1 to about 2%, about 0.2 to about 2%, about 0.3 to about 2%, about 0.5 to about 2%, about 1 to about 2%, about 0.01 to about 1.5%, about 0.05 to about 1.5%, about 0.1 to about 1.5%, about 0.2 to about 1.5%, about 0.3 to about 1.5%, about 0.5 to about 1.5%, about 1 to about 1.5%, about 0.01 to about 1%, about 0.05 to about 1%, about 0.1 to about 1%, about 0.2 to about 1%, about 0.3 to about 1%, about 0.5 to about 1%, about 0.01 to about 0.5%, about 0.05 to about 0.5%, about 0.1 to about 0.5%, about 0.2 to about 0.5%, or about 0.3 to about 0.5%. In certain embodiments, AAV viral particles having a mispackaged HSV vector sequence is undetectable.

With the general principles of the invention set forth herein, the sections below provides further detailed description for various aspects of the invention. It should be understood that any embodiment of the invention can be combined with any one or more additional embodiments of the invention, including those embodiments described in different sections of the application, and those described only in the examples, drawings, or claims.

### 2. Recombinant Replication-Defective Virus

In one aspect, the invention described herein provides a recombinant replication-defective virus derived from the Herpesvirales order (such as Herpes Simplex Virus or HSV), wherein the virus is characterized by a defective ICP27 gene or a functional equivalent gene thereof (such as a deletion in ICP27) that impairs or otherwise does not support replication of the virus, wherein in the case of ICP27 deletion, the deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of said gene encoding ICP27 (e.g., SEQ ID NO: 2), or the functional equivalent gene thereof. The virus (such as the recombinant replication-defective HSV) is further characterized by having a gene of interest (GOI) flanked by AAV ITR sequences, inserted at or within a locus non-essential for viral replication, such as the UL43 locus of HSV or a functional equivalent locus thereof, or a replaceable essential locus such as UL54, and, having an expression cassette comprising a coding sequence for AAV Rep and Cap proteins, again inserted at or within the locus non-essential for viral replication, such as the UL43 locus of the HSV or the functional equivalent locus thereof, or a replaceable essential locus such as UL54.

In certain embodiments, the recombinant replication-defective virus (e.g., HSV) is not a clinical strain/non-laboratory strain of HSV.

In certain embodiments, the recombinant replication-defective virus (e.g., HSV) is a laboratory strain of HSV, such as KOS, KOS 1.1, KOS 1.1A, KOS63, KOS79, McKrae, Stain 17, F17, McIntyre, or viruses resulting from reconstitution of HSV BAC E. coli DNAs, such as BAC HSV KOS-37 or BAC HSV 17-37 (Gierasch et al., J Virol Meth 135:197-206, 2006) and other BAC HSV DNAs.

As used herein, “clinical strain” and “non-laboratory strain” are used interchangeably herein to refer to viral strains that have been relatively recently or freshly isolated from human or a non-human animal. One key distinction between a laboratory and non-laboratory strain is that laboratory strains have been maintained for long periods (e.g., years in some cases), in culture or serial passage (excluding time spent on storage after freezing down). A laboratory strain that has been through many generations of serial passage in culture may have accumulated mutations that favor rapid replication and growth in culture, but may also have lost certain properties useful for practical applications, such as maintenance of the capacity to travel along axons.

In certain embodiments, a viral vector (e.g., HSV vector) of the invention is derived from a virus strain that has undergone more than three years in culture since isolation of its unmodified clinical precursor strain from its host. The time in culture is time actually spent in culture, excluding storage time after freezing down.

In certain embodiments, a viral vector (e.g., HSV vector) of the invention is derived from a virus strain that has undergone more than 1,000 cycles of serial passage since isolation of its unmodified clinical precursor strain from its host.

Because of the deletion, the recombinant replication-defective virus of the invention is replication defective in the absence of ICP27 protein or a functional equivalent provided in trans by a host cell.

The ICP27 (Infected cell protein 27) gene in human herpesvirus 1 (HHV-1) (Human herpes simplex virus 1) encodes a 512-amino acid protein (UniProtKB—Q3MU88 (Q3MU88_HHV1), incorporated herein by reference). It is also known as mRNA export factor, Immediate-early protein IE63, VMW63, and UL54. A sequence of the ICP27 gene is provided in SEQ ID NO: 2, including the native promoter sequence from HSV-1.

The subject recombinant replication-defective virus can be derived from any virus of the Herpesvirales order, which virus may carry a functional equivalent gene of ICP27 from HSV-1. The deletion of the ICP27 gene or its functional equivalent is at least 1,200 bps in length, and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp (i.e., leave nothing) of the most 3′-end of said gene encoding ICP27 (e.g., SEQ ID NO: 2), or the functional equivalent gene thereof.

The genus Herpesvirus was established in 1971 in the first report of the International Committee on Taxonomy of Viruses (ICTV). This genus consisted of 23 viruses and 4 groups of viruses. In the second ICTV report in 1976, this genus was elevated to family level the Herpetoviridae. Because of possible confusion with viruses derived from reptiles, this name was changed in the third report in 1979 to Herpesviridae. In this report, the family Herpesviridae was divided into 3 subfamilies (Alphaherpesvirinae, Betaherpesvirinae and Gammaherpesvirinae) and 5 unnamed genera: 21 viruses were listed. In 2009, the family Herpesviridae was elevated to the order Herpesvirales. This elevation was necessitated by the discovery that the herpesviruses of fish and molluscs were only distantly related to those of birds and mammals. Two new families were created—the family Alloherpesviridae which incorporates bony fish and frog viruses and the family Malacoherpesviridae which contains those of molluscs.

The functional equivalent genes from known viruses of the Herpesvirales order, including those from the various named families, subfamilies, genus and species, can be readily obtained from public or proprietary databases, such as GenBank, UniPro, EMBL, etc., using the human HSV-1 ICP27 polynucleotide sequence (such as SEQ ID NO: 2) as a query. Thus, these sequences are not described herein, but are otherwise incorporated herein by reference.

In certain embodiments, the most 3′-end of the ICP27 gene or functional equivalent gene thereof is defined by the nucleotide immediately 5′ to the next gene on the respective viral genome (e.g., the first nucleotide of the promoter of the “next” gene 3′ to the ICP27 gene or equivalent.).

In certain embodiments, the most 3′-end of the ICP27 gene or functional equivalent gene thereof is defined by the stop codon of ICP27 or the functional equivalent gene thereof, including the stop codon itself.

In certain embodiments, the gene encoding ICP27 has the polynucleotide sequence of SEQ ID NO: 2.

In certain embodiments, the deletion is at least 1,300 bp, 1,400 bp, 1,500 bp, 1,600 bp, 1,700 bp, 1,800 bp, 1,900 bp, 2,000 bp, 2,100 bp or more.

In certain embodiments, the deletion comprises, consisting essentially of, or consisting of the entire coding sequence (or ORF) of the gene encoding ICP27 or the functional equivalent gene thereof.

In certain embodiments, the deletion further comprises the entire promoter region of the gene encoding ICP27 or the functional equivalent gene thereof, or a portion (e.g., the most 3′ about 400 nucleotides) of the promoter region.

In certain embodiments, the virus is derived from the Alloherpesviridae family or the Malacoherpesviridae family.

In certain embodiments, the virus is derived from the Herpesviridae family, such as the Alphaherpesvirinae subfamily, the Betaherpesvirinae subfamily, or the Gammaherpesvirinae subfamily.

In certain embodiments, the virus is derived from HHV-1 (Herpes Simplex Virus-1 or HSV-1), HHV-2 (Herpes Simplex Virus-2 or HSV-2), HHV-3 (Varicella Zoster Virus or VZV), HHV-4 (Epstein-Barr Virus or EBV), HHV-5 (Cytomegalovirus or CMV), HHV-6A/HHV-6B (Roseolovirus, Herpes Lymphotropic Virus), HHV-7, or HHV-8 (Kaposi's Sarcoma-Associated Herpesvirus or KSHV).

In certain embodiments, the virus is derived from Cercopithecine herpesvirus-1 (CeHV-1) or Murid Herpesvirus 68 (MHV-68 or MuHV-4).

In certain embodiments, the virus is derived from porcine Alpha-herpesviruses, including pseudorabies virus (PRV).

In certain embodiments, the virus is derived from the Simplexvirus genus, e.g., Ateline herpesvirus 1, spider monkey herpesvirus, Porcine herpesviruses, Bovine herpesvirus 2, Cercopithecine herpesvirus 1 (Herpes B virus), Fruit bat alphaherpesvirus 1, Macacine herpesvirus 1, Leporid herpesvirus 4, Macropodid herpesvirus 2, and Papiine herpesvirus 2.

In certain embodiments, the virus is derived from the Varicellovirus genus, such as Bovine herpesvirus 1, Bovine herpesvirus 5, Bubaline herpesvirus 1, Caprine herpesvirus 1, Canine herpesvirus 1, Cercopithecine herpesvirus 9, Cervid herpesvirus 1, Cervid herpesvirus 2, Elk herpesvirus 1, Equine herpesvirus 1, Equine herpesvirus 3, Equine herpesvirus 4. Equine herpesvirus 8, Equine herpesvirus 9, Feline herpesvirus 1, and Suid herpesvirus 1.

In certain embodiments, the virus is derived from the Mardivirus genus, such as Anatid herpesvirus 1, Columbiform herpesvirus 1, Gallid herpesvirus 2, Gallid herpesvirus 3 (GaHV-3 or MDV-2), Meleagrid herpesvirus 1 (HVT), and Peacock herpesvirus 1.

In certain embodiments, the virus is derived from the Litovirus genus, such as Gallid herpesvirus 1, and Psittacid herpesvirus 1.

In certain embodiments, the virus is derived from a reptilian Alphaherpesvirus, such as Caretta herpesvirus, Chelonid herpesvirus 1, Chelonid herpesvirus 2, Chelonid herpesvirus 3, Chelonid herpesvirus 4, Chelonia mydas herpesvirus, Coober herpesvirus, Emydid herpesvirus 1, Emydid herpesvirus 2, Fibropapilloma associated herpes virus, Gerrhosaurid herpesvirus 1, Gerrhosaurid herpesvirus 2, Gerrhosaurid herpesvirus 3, Glyptemis herpesvirus 1, Glyptemys herpesvirus 2, Iguanid herpesvirus 1, Iguanid herpesvirus 2. Loggerhead orocutaneous herpesvirus, Lung-eye-trachea associated herpesvirus, Pelomedusid herpesvirus 1, Red eared slider herpes virus, Terrapene herpesvirus 1. Terrapene herpesvirus 2, Testudinid herpesvirus 1. Testudinid herpesvirus 2, Testudinid herpesvirus 3, Testudinid herpesvirus 4, and Varanid herpesvirus 1.

In certain embodiments, the virus is derived from the Rhadinovirus genus, such as Alcelaphine herpesvirus 1, Alcelaphine herpesvirus 2, Ateline herpesvirus 2, Bovine herpesvirus 4, Cercopithecine herpesvirus 17, Equine herpesvirus 2, Equine herpesvirus 5, Equine herpesvirus 7, Japanese macaque rhadinovirus, Leporid herpesvirus 1, and Murid herpesvirus 4 (Murine gammaherpesvirus-68 or MHV-68).

In certain embodiments, the virus is a strain of HSV-1, such as KOS, KOS 1.1, KOS 1.1A, KOS-37, KOS63, KOS79, McKrae, Stain 17, F17, Mcintyre, or viruses resulting from reconstitution of HSV BAC E. coli DNAs, such as BAC HSV KOS-37 or BAC HSV 17-37 (Gierasch et al., J Virol Meth 135:197-206, 2006) and other BAC HSV DNAs.

In certain embodiments, the functional equivalent gene thereof is ORF57 of KSHV, Mta/SM/EB2 of EBV. UL69 of human CMV, or other equivalent genes in any viruses from the Herpesvirales order.

In certain embodiments, the coding sequence for the AAV Rep and Cap proteins, and the gene of interest (GOI) flanked by AAV ITR sequences, are integrated into or replaces a non-essential gene of the virus (e.g., not required for viral replication and not required for viral packaging). Exemplary such non-essential genes include the TK gene at the UL23 locus, the UL43 locus, the US5 locus, and most of the other about 50% of the viral genome.

In certain embodiments, the locus non-essential for viral replication is not the TK locus/UL23, to enable the use of approved drugs for HSV.

In certain embodiments, the locus non-essential for viral replication is UL43.

In certain embodiments, the defective ICP27 gene comprises/is a complete deletion of the UL54/ICP27 locus of the HSV or a functional equivalent thereof (e.g., a complete deletion of SEQ ID NO: 2, or a coding sequence for SEQ ID NO: 1).

In certain embodiments, the recombinant replication defective virus (e.g., rHSV) comprises an intact HSV UL42 locus (or functional equivalent thereof) and an intact UL44 locus (or functional equivalent thereof). Optionally, the recombinant replication defective virus (e.g., rHSV) further comprises an intact UL23 locus (or functional equivalent thereof) and/or an intact US5 locus (or functional equivalent thereof).

In certain embodiments, the GOI is between UL42 and the coding sequence for AAV Rep/Cap.

In certain embodiments, the GOI is between UL44 and the coding sequence for AAV Rep/Cap.

Another aspect of the invention provides a method of propagating/amplifying/producing the recombinant replication-defective virus of the invention, the method comprising infecting the subject host cell expressing a complementary/functional ICP27 gene or a functional equivalent thereof (see below), with the subject recombinant replication-defective virus.

In certain embodiments, the method further comprises harvesting the recombinant replication-defective virus from the infected host cell.

In certain embodiments, there is no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, or 2 bp sequence overlap between the subject recombinant replication-defective virus, and the coding sequence for the ICP27 or the functional equivalent thereof which may be integrated into the host cell genome.

The subject recombinant replication-defective virus derived from the Herpesvirales order can be made or constructed using conventional molecular biology techniques, such as homologous recombination. For example, to delete the native ICP27 gene or coding sequence from a wild-type strain of HSV (or a functional equivalent gene from a virus of the Herpesvirales order), the genome of the wild-type virus can be inserted into a suitable vector, such as a bacterial artificial chromosome (BAC) or a yeast artificial chromosome (YAC). Homologous recombination can then be carried out in a suitable host cell, such as an E. coli, a yeast, an insect cell (e.g., SF9), or a mammalian cell, to delete the target gene (i.e., the ICP27 gene or its functional equivalent). This can be done by, for example, introducing into the same host cell a linearized plasmid carrying homologous regions that flank the target gene (i.e., ICP27 or functional equivalent thereof).

Suitable mammalian host cell includes: a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or any other suitable mammalian cells.

Thus another aspect of the invention provides a method of making a recombinant replication-defective virus derived from the Herpesvirales order, wherein the virus is characterized by a deletion in a gene encoding ICP27, or a functional equivalent gene thereof, wherein the deletion is at least 1,200 bps in length and leaves no more than 300 bp, 250 bp, 200 bp, 150 bp, 100 bp, 50 bp, 30 bp, 20 bp, 10 bp, 9 bp, 8 bp, 7 bp, 6 bp, 5 bp, 4 bp, 3 bp, 2 bp, 1 bp or 0 bp of the most 3′-end of the gene encoding ICP27 (e.g., SEQ ID NO: 2), or the functional equivalent thereof, the method comprising creating the deletion of the gene encoding ICP27 or functional equivalent thereof by homologous recombination in a suitable host cell.

In certain embodiments, the homologous recombination is carried out by using a bacterial artificial chromosome (BAC) comprising the genome of the virus derived from the Herpesvirales order (e.g., HSV genome) having the gene encoding ICP27 or the functional equivalent thereof.

In certain embodiments, the cell for amplification of the subject rHSV DNA can be chosen from a large group of cell types, such as an E. coli, or a eukaryotic cell such as a yeast, an insect cell (e.g., SF9), or a mammalian cell. Propagation of the rHSV virus can be conducted in a mammalian cell, e.g., a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others.

### 3. Gene of Interest (GOI) in rAAV and Treatable Diseases

The recombinant replication-defective virus of the invention, such as rHSV vectors of the invention, can be used for large scale production of recombinant AAV vectors carrying a gene of interest (GOI). The GOI can be any gene or coding sequence within the packaging capacity of the AAV, e.g., about 4-5 kb, or about 4.7 kb including the ITR sequences, or about 4.4 kb without accounting for the ITR sequences.

In certain embodiments, the rAAV carrying the GOI can be used in gene therapy to treat a disease or condition caused by lacking of function of an endogenous gene in the host, such as a defective version of the GOI.

As used herein, “gene of interest” or GOI or goi generally refers to a nucleic acid or polynucleotide sequence, such as a gene, an open reading frame (ORF), or a coding sequence for protein or RNA, such as non-coding RNA that includes siRNA, miRNA, shRNA, antisense RNA or a precursor thereof. However, in certain circumstances or context, the term GOI also loosely refers to a protein (encoded by the GOI), or a disease or indication that can be remedied by the GOI, or a disease or indication can be (but is not necessarily) caused by loss of function of the GOI.

For example, the gene GALGT2 encodes the protein GalNAc transferase (β-1,4-N-acetylgalactosamine galactosyltransferase), which is an enzyme that transfers a complex sugar molecule onto a few specific proteins, including dystroglycan. Under normal circumstances, GalNAc transferase is found only at the neuromuscular junction (NMJ), where some components of the dystroglycan-associated protein complex are different than elsewhere in muscle. Importantly, at the NMJ, utrophin is present instead of dystrophin. In the mdx mouse model of muscular dystrophy, viral gene transfer of GALGT2 results in expression of GalNAc transferase across the entire muscle membrane, instead of just at the normal expression domain of the NMJ, as well as upregulation of utrophin across the entire muscle fiber. In the mdx mouse, this expression can correct muscle functional deficits to the same degree as does microdystrophin gene expression. Furthermore, overexpression of GALGT2 corrects muscle pathology in mouse models of other muscular dystrophies, including LGMD2A and congenital muscular dystrophy (MDC1A). Thus GALGT2 is a GOI for treating muscular dystrophy such as DMD, BMD, LGMD2A and MDC1A, even though GALGT2 is not necessarily defective per se in the patient in need of treatment.

In another example, Sarcolipin (SLN) inhibits the sarco/endoplasmic reticulum (SR) Ca²⁺ ATPase (SERCA), and is abnormally elevated in the muscle of DMD patients and animal models such as the mdx mouse model of DMD. Reducing SLN levels by AAV9-mediated RNA interference ameliorates dystrophic pathology in the severe dystrophin/utrophin double mutant (mdx:utr^{-/-}) mouse model of DMD, including attenuation of muscle pathology and improvement of diaphragm, skeletal muscle and cardiac function. Thus the coding sequence for SLN RNAi is a GOI that remedies DMD.

Thus the GOI can be a gene (or protein) that, when expressed, replaces a mutated, damaged, or inactive gene or protein. The GOI can be a gene (or protein) that, when expressed, assists an already functioning process that requires modification for therapy in a disease, disorder, or dysfunction. The GOI can be a gene (or protein) that, when expressed, assists a dysfunctional process that requires modification for therapy in a disease, disorder, or dysfunction. A GOI nucleic acid sequence can be DNA, RNA, or synthetic nucleic acid molecule. The GOI can be a protein, an enzyme, a structural protein, a functional protein, or an adaptable protein based on cell function(s). The GOI can provide therapeutic benefit or a treatment modality for a disease, disorder, or dysfunction.

In certain other embodiments, the GOI may be a CRISPR/Cas effector enzyme, such as a Class 2, Type II, IV, V, or VI effector enzyme, including CRISPR-Cas9, Cas 12, Cas 13, etc. In certain embodiments, the GOI may be a TALEN, or other genetic based gene editing protein that are required for intracellular delivery for their intended activity, such as gene editing or gene knockout in a target cell, tissue, or organism/individual.

In certain embodiments, CRISPR/Cas effector enzyme lacks endonuclease activity (dCas, such as dCas9).

In certain embodiments, the Cas or dCas is further fused to a base editor, such as a cytosine base editor (CBE, e.g., APOBEC, BE1, BE2, BE3, Target-AID base editor, SaBE3, BE3 PAM variants, BE3 editing window variants, AID, CDA1, APOBEC3G, HF-BE3, BE4, BE4max, and AncBE4max), an adenine base editor (ABE, e.g., ABE7.10, ABE 6.3, ABE7.8, ABE7.9, ABEmax, ABE8e(TadA-8e V106W), ABE8 and variants thereof), or a dual base editor (SPACE, A&C-BEmax).

Any and all GOIs as used herein may require codon optimization for enhanced expression and activity via known computer based algorithms.

The AAV ITR sequences flanking the GOI can be from any AAV ITR, and can be from the same or different AAV serotypes. In certain embodiments, the AAV ITR sequences flanking the GOI are both from AAV1, AAV2, AAV3A, AAV3B, AAV4, AAV5, AAV6, AAV7, AAV8, AAV9, AAV10, AAV11, AAV12, AAV13, or AAV-DJ.

In certain embodiments, the tropism of the AAV include skeletal muscle (such as AAV1, AAV6, AAV7, AAV8, or AAV9, preferably AAV9).

In certain embodiments, the AAV ITR, the AAV Rep, and the AAV Cap are from the same or different AAVs.

In certain embodiments, the AAV ITR is AAV2 ITR, the AAV Rep is Rep2 from AAV2, and the AAV Cap is Cap9 from AAV9.

Thus the rAAV that may be produced by using the subject viral vectors (e.g., rHSV vectors) and the complementary cells (which supply the ICP27 gene product in trans), may encode a gene of interest (GOI) useful for, e.g., gene therapy to treat a disease or condition. Representative (non-limiting) gene of interest (GOI) may include: a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is any one described in the following patents: U.S. Pat. Nos. 7,906,111; 7,001,761; 7,510,867; 6,869,777; 8,501,920; 7,892,824; PCT/US2016/013733; U.S. Pat. No. 10,166,272 (all incorporated herein by reference). In certain embodiments, the microdystrophin gene is capable of being packaged into a rAAV virion, e.g., no more than about 4.7 kb in size.

In certain embodiments, the microdystrophin gene contains within its coding sequence spectrin-like repeats R16 and R17 that are capable of restoring nitric oxide synthase (nNOS) activity to the sarcolemma (such as those described in U.S. Pat. No. 7,892,824).

In certain embodiments, the microdystrophin gene comprises a coding sequence for the R1, R16, R17, R23, and R24 spectrin-like repeats (i.e., SR1, SR16, SR17, SR23, and SR24, respectively) of the full-length dystrophin protein, such as one described in PCT/US2016/013733 (incorporated herein by reference). In certain embodiments, the microdystrophin gene does not encode any other spectrin repeats of the full-length dystrophin protein, other than SR1, SR16, SR17, SR23, and SR24.

In certain embodiments, the coding sequence for AAV Rep and Cap proteins is under the transcriptional control of a promoter, such as an AAV p5 promoter, an upstream HSV promoter, a modified p5 promoter lacking RBE (Rep-Binding Element), or a ubiquitous promoter (such as CMV promoter, EF1a promoter, CAG promoter, CB promoter etc).

In certain embodiments, the recombinant replication defective viral vector (e.g., rHSV vector) of the invention further comprises a removable transcription unit inserted at or within the UL43 locus of the HSV or functional equivalent thereof, wherein the removable transcription unit comprises a selection marker (such as an antibiotic resistance gene, including but not limited to Kanamycin resistance gene Kan^{R} or zeocin resistance gene Zeo^{R}) under the transcriptional control of a selection marker promoter (such as a Kan^{R} or Zeo^{R} promoter).

In certain embodiments, the removable transcription unit is flanked by a pair of flippase recognition target (FRT) sites.

Diseases or conditions having a potential to benefit from the rAAV produced by the rHSV-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, A1At Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease.

In certain embodiments, diseases or conditions having a potential to benefit from the rAAV produced by the rHSV-based system may include: Becker muscular dystrophy (BMD), Congenital muscular dystrophies (CMD), Bethlem CMD, Fukuyama CMD, Muscle-eye-brain diseases (MEBs), Rigid spine syndromes, Ullrich CMD, Walker-Warburg syndromes (WWS), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), Motor neuron diseases including ALS (amyotrophic lateral sclerosis), Spinal-bulbar muscular atrophy (SBMA), Spinal muscular atrophy (SMA).

In certain embodiments, diseases or conditions having a potential to benefit from the rAAV produced by the rHSV-based system may include ion channel diseases, which are typically marked by muscular weakness, absent muscle tone, or episodic muscle paralysis. They include Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia.

In certain embodiments, diseases or conditions having a potential to benefit from the rAAV produced by the rHSV-based system may include mitochondrial diseases, which occur when structures that produce energy for a cell malfunction. Such diseases include: Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO).

In certain embodiments, diseases or conditions having a potential to benefit from the rAAV produced by the rHSV-based system may include myopathies, which is a disease of muscle in which the muscle fibers do not function properly, resulting in muscular weakness. Myopathies include: Cap myopathies, Centronuclear myopathies, Congenital myopathies with fiber type disproportion, Core myopathies, Central core disease, Multiminicore myopathies, Myosin storage myopathies, Myotubular myopathy, Nemaline myopathies, Distal myopathies, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, Vocal cord and pharyngeal distal myopathy, Welander distal myopathy, Endocrine myopathies, Hyperthyroid myopathy, Hypothyroid myopathy, Inflammatory myopathies, Dermatomyositis, Inclusion-body myositis, Polymyositis, Metabolic myopathies, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), Myofibrillar myopathies (MFM), Scapuloperoneal myopathy.

In certain embodiments, diseases or conditions having a potential to benefit from the rAAV produced by the rHSV-based system may include neuromuscular junction diseases, which result from the destruction, malfunction or absence of one or more key proteins involved in the transmission of signals between muscles and nerves. Such diseases include: Congenital myasthenic syndromes (CMS), Lambert-Eaton myasthenic syndrome (LEMS), Myasthenia gravis (MG).

In certain embodiments, diseases or conditions having a potential to benefit from the rAAV produced by the rHSV-based system may include peripheral nerve diseases, in which the motor and sensory nerves that connect the brain and spinal cord to the rest of the body are affected, causing impaired sensations, movement or other functions. Such diseases include: Charcot-Marie-Tooth disease (CMT), Giant axonal neuropathy (GAN), muscle wasting in cachexia and aging.

### 4. Complementary Recombinant Vectors

The viral vectors of the invention, such as the recombinant HSV vectors of the invention, can be propagated in a suitable host cell that provides the ICP27 function deleted from the subject viral vectors. Such ICP27 function can be provided by the subject complementary recombinant vectors (or “recombinant vector” for short) encoding ICP27. The complementary recombinant vectors of the invention may be integrated into the genome of the host cell. The ICP27 coding sequence may be transcribed from the native promoter in the Herpesvirales genome from which the ICP27 gene originates.

Thus another aspect of the invention provides a (complementary) recombinant vector capable of expressing ICP27 or a functional equivalent thereof in a host cell, the vector comprising: (1) a coding sequence for the ICP27 or the functional equivalent thereof, operatively linked to a promoter capable of directing the transcription of the coding sequence in the host cell; (2) a polyadenylation site 3′ to the coding sequence; and, (3) optionally, one or more multi-cloning site(s); wherein the vector contains no more than 300 bp, 250 bp, 200 bp, 150 bp, 100 bp, 50 bp, 30 bp, 20 bp, 10 bp, 9 bp, 8 bp, 7 bp, or 6 bp consecutive nucleotides of any of the subject recombinant replication-defective virus (e.g., rHSV).

Since the ICP27 protein provided in trans does not need to be 100% as active as the wild-type ICP27, in certain embodiments, the ICP27 has the amino acid sequence of SEQ ID NO: 1, or is at least 60%, 70%, 75%, 80%, 85%, 90%, 95%, 97%, 98%, 99%, 99.2%, 99.4%, 99.6%, or 99.8% identical to SEQ ID NO: 1.

Likewise, the promoter of the ICP27 coding sequence does not need to be the native promoter in the virus from which the ICP27 originates, though in some embodiment, the promoter is the native promoter.

In certain embodiments, the promoter comprises at least about 400 polynucleotides, 450 polynucleotides, 500 polynucleotides, or about 550 polynucleotides.

In certain embodiments, the promoter comprises nucleotides 1-538 of SEQ ID NO: 2, or nucleotides 127-538 of SEQ ID NO: 2, or nucleotides 113,139-113,550 of GenBank Accession No. KT887224, or nucleotides 113,013-113,550 of GenBank Accession No. KT887224 (entire sequence incorporated herein by reference).

In certain embodiments, there is no overlap in sequence between the ICP27 coding sequence on the complementary recombinant vectors of the invention, and the deleted ICP27 coding sequence from the subject viral vectors.

In certain embodiments, the ICP27 coding sequence as the complementary DNA in the host cell (useful for the propagation of the subject viral (e.g., rHSV) vectors devoid of ICP27 coding sequence) is partially or fully codon-optimized for translation in a eukaryote or mammalian cell line, such as in BHK cells, Vero cells, or HEK293 cells. For example, in certain embodiments, the most 3′ 300-350 nucleotides of the coding sequence are codon-optimized for expression in the mammalian host cell.

In certain embodiments, the polyadenylation site is a bovine growth hormone (bGH) polyadenylation site.

In certain embodiments, the polyadenylation site or poly(A) signal sequence is from other suitable sources, e.g., synthetic sequences or sequences from other eukaryotic genes or viruses.

In certain embodiments, any minimal or residual overlap between the ICP27 coding sequence on the complementary recombinant vectors of the invention, and the subject recombinant replication-defective viral vector without the deleted ICP27 coding sequence/ORF, is not significant enough to permit or support homologous recombination between the two. For example, there is minimal (if any) residue ICP27 coding sequence and/or promoter sequence on the subject viral vector, such that any overlap in sequence between the subject viral vector and the ICP27 coding sequence (plus any native promoter sequence) is insufficient to lead to homologous recombination.

In certain embodiments, the ICP27 coding sequence as the complementary DNA in the host cell (useful for the propagation of the subject (e.g., rHSV) vectors devoid of ICP27 coding sequence) comprises a mutation that reduce the ICP27 protein's ability to inhibit host pre-mRNA splicing, while still allowing the promotion of late gene expression. Such ICP27 mutation may lead to a greater infectious rAAV yield due at least partly to increased expression of the AAV Rep and Cap proteins.

Exemplary such mutations include the vBS3.3, vBS4.3, and vBS5.3 mutations as described by Soliman et al. (J Virol 71:9188-9197, 1997, incorporated herein by reference). Specifically, Soliman described the use of a temperature sensitive ICP27 allele—LG4—that loses ICP27 activity at the restrictive temperature of 39.5° C., in a genetic screen for intragenic suppressors. Three such intragenic suppressors were identified, namely vBS3.3, vBS4.3, and vBS5.3. The LG4 allele has a single R480H point mutation just N-terminal to the carboxy-terminal zinc finger in wild-type ICP27. The vBS3.3, vBS4.3, and vBS5.3 intragenic suppressor alleles have all retained the original R480H point mutation, but also contain one additional point mutation of V496I, S334L, and V487I, respectively. Thus the vBS3.3 is a double point mutation of R480H and V496I. The vBS4.3 is a double point mutation of R480H and S334L. The vBS5.3 is a double point mutation of R480H and V487I.

### 5. Host Cells

One aspect of the invention comprises a host cell comprising the recombinant replication defective viral vector (e.g., rHSV vector) of the invention.

In certain embodiments, the host cell does not comprise a coding sequence for ICP27, and which optionally further comprises helper virus proteins required for AAV packaging. In certain embodiments, the host cell is a HEK293 cell (such as an Expi293F cell), a HeLa cell, an A549 cell, a BHK cell, or an insect cell (such as Sf9).

In certain other embodiments, the host cell comprises a coding sequence for, and is capable of expressing ICP27 or a functional equivalent thereof. For example, in certain embodiments, the host cell comprises: (1) a coding sequence for said ICP27 or said functional equivalent thereof, operatively linked to a promoter capable of directing the transcription of said coding sequence in the host cell; (2) a polyadenylation site 3′ to the coding sequence; and, (3) optionally, one or more multi-cloning site(s).

Many different types of eukaryote host cells can be used to propagate the subject recombinant replication-defective viral vectors, provided that such eukaryote host cells are engineered to express ICP27 deleted from the subject recombinant replication-defective viral vectors.

In certain embodiments, the subject host cells comprise the subject complementary recombinant vectors, and are capable of expressing ICP27 to promote replication and packaging of the subject viral vectors.

In certain embodiments, the ICP27 has the amino acid sequence of SEQ ID NO: 1, or is at least 60%, 70%, 75%, 80%, 85%, 90%, 95%, 97%, 98%, 99%, 99.2%, 99.4%, 99.6%, or 99.8% identical to SEQ ID NO: 1.

In certain embodiments, the promoter comprises nucleotides 1-538 of SEQ ID NO: 2, nucleotides 127-538 of SEQ ID NO: 2, nucleotides 113,139-113,550 of GenBank Accession No. KT887224, or nucleotides 113,013-113,550 of GenBank Accession No. KT887224.

In certain embodiments, the coding sequence is partially or fully codon-optimized for expression in a mammalian host cell. For example, the most 3′ 300-350 nucleotides of the coding sequence may be codon-optimized for expression in the mammalian host cell.

In certain embodiments, said polyadenylation site is a bovine growth hormone (bGH) polyadenylation site.

In certain embodiments, the coding sequence for said ICP27 comprises a mutation that reduces inhibition of host cell pre-mRNA splicing, while permitting HSV late gene expression. In certain embodiments, the mutation is vBS3.3 double mutation, vBS4.3 double mutation, or vBS5.3 double mutation.

In certain embodiments, the recombinant vector is stably integrated into the host cell genome.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell. Human cells include BHK cells, Vero cells, HEK293 cells, etc.

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin. For growing adherent HEK293 cells, the percentage of FBS can be reduced during rAAV production in order to limit contamination by animal-derived components.

In certain embodiments, the host cell is a Vero cell, such as a Vero75.4 or V75 cell described herein. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21 or sBHK27. In certain embodiments, the BHK cells are adapted to grow in serum-free suspension.

In certain embodiments, the host cell is a HEK293 cell. In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference).

In certain embodiments, the HEK293 cell is a HEK293T cell which expresses SV40 T antigen (the temperature sensitive allele tsA1609) and the neomycin/geneticin-resistance gene.

In certain embodiments, the cell for amplification of the subject rHSV DNA can be chosen from a large group of cell types, such as an E. coli, or a eukaryotic cell such as a yeast, an insect cell (e.g., SF9), or a mammalian cell. Propagation of the rHSV virus can be conducted in a mammalian cell, e.g., a Vero cell, a baby hamster kidney (BHK) cell, a HeLa cell, a human lung fibroblast MRC-5, a human foreskin fibroblast (HFF), a human embryonic lung fibroblast (HELF), a Madin-Darby canine Kidney cell (MDCK), a Madin-Darby bovine kidney cell (MDBK), or others.

In certain embodiments, stocks of viral vectors so propagated can be checked to ensure that no replication competent viral vectors are present. For example, assays used in Example 1 can be used to determine the titer of rHSV, and the presence or absence rcHSV.

In certain embodiments, the viral vectors of the invention may be adapted for use in producing recombinant AAV vectors encoding a gene of interest (GOI), which may be used in gene therapy. See the section entitled “Recombinant AAV Production” below. In such embodiments, one or more rAAV production cell lines may be infected by the subject viral vectors, such as rHSV vector encoding AAV Rep and Cap proteins, and optionally rHSV vector encoding the GOI flanked by AAV ITR sequences.

In certain embodiments, such producer cell line for rAAV production is a HeLa- or A549-derived cell line transfected with a plasmid containing both rep-cap genes of AAV, and an rAAV vector genome (with the GOI) along with a drug selection marker.

In certain embodiments, such producer cell line for rAAV production is a Vero cell.

In certain embodiments, such producer cell line for rAAV production is a BHK cell.

In certain embodiments, such producer cell line for rAAV production is a HEK293 cell.

In certain embodiments, such producer cell line for rAAV production comprises an rAAV provirus that encodes the GOI flanked by the AAV ITR sequences, wherein the rAAV provirus is integrated into the genome of the producer cell line for rAAV production. The GOI can be any one of the GOI described herein useful for gene therapy, such as a dystrophin minigene or a microdystrophin gene described in U.S. Pat. Nos. 7,906,111; 7,001,761; 7,510,867; 6,869,777; 8,501,920; 7,892,824; or U.S. Pat. No. 10,166,272, or in PCT/US2016/013733 (all incorporated herein by reference).

For example, PCT/US2016/013733 (WO2016/115543A2) provides a micro-dystrophin gene operatively connected to a regulatory cassette, wherein the micro-dystrophin gene encodes a protein comprising: an amino-terminal actin-binding domain; a β-dystroglycan binding domain; and a spectrin-like repeat domain, comprising at least four spectrin-like repeats, wherein two of the at least four spectrin-like repeats comprise a neuronal nitric oxide synthase binding domain. In certain embodiments, the at least four spectrin-like repeats include spectrin-like repeat 1 (SR1), spectrin-like repeat 16 (SR16), spectrin-like repeat 17 (SR17), and spectrin-like repeat 24 (SR24). In certain embodiments, the protein encoded by the micro-dystrophin gene further comprises at least a portion of a hinge domain, such as at least one of a Hinge 1 domain, a Hinge 2 domain, a Hinge 3 domain, a Hinge 4 domain, and a hinge-like domain. In certain embodiments, the micro-dystrophin gene comprises, in N- to C-terminal order: a Hinge 1 domain (H1); a spectrin-like repeat 1 (SR1); a spectrin-like repeat 16 (SR16); a spectrin-like repeat 17 (SR17); a spectrin-like repeat 24 (SR24); and a Hinge 4 domain (H4). In certain embodiments, H1 is directly coupled to the SR1. In certain embodiments, SR1 is directly coupled to SR16. In certain embodiments, SR16 is directly coupled to SR17. In certain embodiments, SR 17 is directly coupled to SR24. In certain embodiments, SR24 is directly coupled to the H4. In certain embodiments, the protein encoded by the micro-dystrophin gene further comprises between SR1 and SR16, in N- to C-terminal order, a spectrin-like repeat 2 (SR2) and a spectrin-like repeat 3 (SR3). In certain embodiments, SR1 is directly coupled to SR2 and SR2 is further coupled to SR3. In certain embodiments, H1 is directly coupled to SR1. SR1 is directly coupled to SR16, SR16 is directly coupled to SR17, SR17 is directly coupled to SR23, SR23 is directly coupled to SR24, and SR24 is directly coupled to H4.

In certain embodiments, the regulatory cassette is selected from the group consisting of a CK8 promoter and a cardiac troponin T (cTnT) promoter. In certain embodiments, the protein encoded by the micro-dystrophin gene has between five spectrin-like repeats and eight spectrin-like repeats. In certain embodiments, the protein encoded by the micro-dystrophin gene has at least 80% or 90% sequence identity to the amino acid sequence of SEQ ID NO: 4 or 5 in WO2016/115543A2 (incorporated herein by reference).

In certain embodiments, rAAV viral vectors so produced can be checked to ensure that no rHSV and no rcHSV viral vectors are present in the rAAV viral stock. For example, assays used in Example 1 can be used to determine the presence or absence rHSV and rcHSV.

### 6. Recombinant HSV and/or AAV Production

The recombinant replication-defective viral vectors of the present invention, especially the A-I-O recombinant HSV vectors of the present invention, as well as the production cell lines, together form an HSV-based complementation system that can be used for large scale production of recombinant AAV vectors (rAAV) useful for gene therapy. In certain embodiments, the system and the associated method of use, can be used for simultaneous propagating/amplifying/producing of AAV viral particles comprising a GOI, as well as the subject A-I-O HSV vector comprising the ITR-flanked GOI and rep/cap coding sequences required for AAV packaging/production, with the method comprising infecting a suitable subject host cell (e.g., one with ICP-27 expression) with the recombinant replication-defective HSV viral particle of the invention.

Recombinant AAV vectors, which can be produced with the present viral (e.g., rHSV) vectors and production cell lines, typically comprise a gene of interest (GOI) and expression regulators (such as promoters for the GOI) in lieu of the wild-type AAV virus rep and cap open reading frames (ORFs). The AAV rep and cap ORFs, optionally their native promoters p5, p19, and p40 and other AAV packaging required helper functions, are instead supplied by the subject recombinant HSV vector and/or production cell line. The rep ORF encodes four nonstructural Rep proteins involved in the AAV viral life cycle, and the cap ORF encodes the three structural proteins (i.e., VP1, VP2, and VP3) that form the icosahedral AAV capsid. Both the rep and cap coding sequences are provided by the subject A-I-O HSV vectors. Typically, the only AAV viral sequences that are retained in the rAAV vector genome are the inverted terminal repeats (ITRs)—the minimal cis-acting elements required for AAV DNA replication and packaging.

The gene of interest (GOI) may include genes useful for gene therapy in treating certain diseases or conditions. Representative (non-limiting) GOI may include a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

Suitable microdystrophin genes have been described in the following patents: U.S. Pat. Nos. 7,906,111; 7,001,761; 7,510,867; 6,869,777; 8,501,920; 7,892,824; PCT/US2016/013733; U.S. Pat. No. 10,166,272 (all incorporated herein by reference).

Diseases or conditions having a potential to benefit from the rAAV produced by the subject rHSV-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, A1At Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease, etc.

Being a naturally replication-defective human parvovirus, wild-type AAV integrates its genome site-specifically within the host cell chromosome in the absence of helper assistance for its replication, where it persists indefinitely unless rescued via cellular infection with a helper virus. The introduction of a helper virus into the host cell triggers AAV replication and the generation of progeny virions. In the case of rAAV virions useful for gene therapy, introduction of the helper virus function into a suitable host cell triggers the packaging of the GOI in the rAAV virions, when the requisite rep and cap coding sequences are also supplied in the same system.

In other words, production of recombinant AAV relies on (1) the presence of the AAV rep and cap coding sequences, and (2) the helper virus functions. The present A-I-O recombinant (HSV) vector and production cell line can provide both required functionality for rAAV production.

Viruses of the Herpesviridae families, such as HSV, have been shown to provide the essential trans functions for AAV replication (Handa and Carter, J. Biol. Chem. 254:6603-6610, 1979; Buller et al., J. Virol. 40:241-247, 1981). For simplicity, the description herein refers to HSV as a specific example of a virus from the Herpesviridae family that can provide the essential trans functions for AAV replication, and it should be understood that the description generally applies to other virus from the Herpesvirales order, such as the Herpesviridae family.

Replication proteins from viruses of the Herpesvirales order, such as the Herpesviridae family (e.g., HSV), can be used directly by AAV for efficient genome replication and packaging.

Thus in certain embodiments, the A-I-O rHSV of the present invention can be used to produce rAAV with GOI, by incorporating both the ITR-flanked GOI, as well as the AAV rep and cap coding sequences into the subject rHSV vector. According to this embodiment, the GOI and AAV rep and cap genes can both be inserted into (with or without replacing) a non-essential gene (such as the UL43 locus) of the subject replication-defective rHSV vector with ICP27 deletion at the UL54 locus, by, e.g., homologous recombination. Alternatively, the GOI and AAV rep and cap genes can both be inserted into a replaceable essential gene (such as the UL54 locus) of the subject replication-defective rHSV vector with ICP27 deletion at the UL54 locus, by, e.g., homologous recombination. The resulting rHSV can be propagated in V27-like cells (such as those derived from Vero cells, e.g., the V75.4 or V75 cells) of the invention that contain an ICP27 coding sequence not overlapping (or minimally overlapping) with the subject ICP27-deleted rHSV. The resulting rHSV particles can produce the requisite AAV Rep and Cap proteins when used to infect a suitable AAV production cell line (such as BHK cells, Vero cells, or HEK293 cells).

In other embodiments, recombinant AAV vectors with a desired GOI is produced by infecting an ICP27-expressing cell line (such as V75 and sBHK27) with the A-I-O rHSV of the present invention having AAV rep and cap genes, as well as the recombinant AAV vectors including the GOI flanked by the AAV ITR sequences.

Specifically, the AAV rep and cap coding sequences, and the AAV ITR-flanked GOI may be inserted into the same (or different) non-essential gene locus on the subject rHSV, such as the UL43 locus or the thymidine kinase (TK) locus of the subject replication-defective rHSV vector with ICP27 deletion, by, e.g., homologous recombination. The resulting rHSVs can be propagated in V27-like cells (such as those derived from Vero cells, e.g., V75 or sBHK27 cells) of the invention that contain an ICP27 coding sequence not overlapping (or minimally overlapping) with the subject ICP27-deleted rHSV. The resulting rAAV virions carrying the GOI can be directly isolated/purified from the ICP-27 expressing host cells.

This is a rAAV manufacturing system based solely on HSV infection. In one embodiment, rAAV particles carrying the GOI can be produced by first infecting a suitable rHSV production cell line (such as Vero cells or BHK cells) with the requisite complementation system (such as the ICP27 coding sequence not overlapping with the subject rHSV) to produce rHSV stock. After rHSV vector recovery and concentration to high titer, A-I-O rHSV vectors carrying the AAV rep and cap coding sequences, as well as the AAV ITR-flanked GOI, are used to infect a suitable AAV production cell line, such as HEK293 or BHK cells, to produce rAAV vectors carrying the GOI. In another embodiment, rAAV viral particles can be directly accomplished by infecting the ICP-27 expressing cell line such as V75 or sNHK27.

Thus in another aspect, the invention provide a method of producing a recombinant Adeno-Associated Virus (rAAV) comprising a gene of interest (GOI) coding sequence flanked by AAV ITR sequences, the method comprising infecting an ICP-27-expressing production host cell with a recombinant replication-defective virus comprising a coding sequence for AAV Rep and Cap proteins, and a gene of interest (GOI) flanked by AAV ITR sequences.

In certain embodiments, the production cell line is BHK. Vero, or HEK293.

In certain embodiments, the tropism of the AAV include serotypes such as AAV1, AAV2, AAV6, AAV7, AAV8, or AAV9, AAV10, AAV11, preferably AAV9. In certain embodiments, AAV capsids may be genetically modified, or capsids may be synthetic, designer capsids that enhance tissue specific or physiologic compartments delivery of a GOI to a specific tissue such as muscle, skeletal muscle, cardiac muscle, smooth muscle, and the like, as described (sec, e.g., Zinn and Grimm, High-Throughput Dissection of AAV-Host Interactions: The Fast and the Curious, JMB 430(17):2626-2640, 2018; Kotterman and Schaffer, Engineering adeno-associated viruses for clinical gene therapy. Nature Reviews Genetics (2014) 4445-4451, both incorporated herein by reference). Tropism of AAV through pseudotyping, or the mixing of a capsid and genome from different viral serotypes may also be employed. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, pseudotyped AAV2/5 targets myoblasts (Duan et al., Enhancement of muscle gene delivery with pseudotyped adeno-associated virus type 5 correlated with myoblast differentiation. J Virol 75(16):7662-7671, 2001). Other pseudotyped AAV includes AAV2/6. In certain embodiments, In-silico-derived sequences were synthesized de novo and characterized for biological properties relevant to clinical applications. This effort led to the generation of nine functional putative ancestral AAVs and the identification of Anc80, the predicted ancestor of the widely studied AAV serotypes 1, 2, 8, and 9, as a highly potent in vivo gene therapy vector for targeting liver, muscle, and retina (Zinn et al., In Silico Reconstruction of the Viral Evolutionary Lineage Yields a Potent Gene Therapy Vector, Cell Reports 12(6): 1056-1068, 2015); Buning et al., Engineering the AAV capsid to optimize vector-host-interactions, Current Opinion in Pharmacology, 24:94-104, 2015).

In certain embodiments, the tropism of the AAV include skeletal muscle (such as AAV1, AAV6, AAV7, AAV8, or AAV9, preferably AAV9).

In certain embodiments, the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a functional equivalent of dystrophin (e.g., a dystrophin minigene encoding a functional micro-dystrophin protein).

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the microdystrophin gene does not comprise coding sequence for and spectrin repeats of the full-length dystrophin protein other than the SR1, SR16, SR17. SR23, and SR24 repeats (e.g., in that order).

In certain embodiments, the subject rHSV provides the minimal set of HSV genes required for AAV production, including the HSV core replication machinery—the HSV helicase-primase complex (encoded by UL5, UL8, and UL52), and the single-stranded DNA-binding protein encoded by UL29—as well as other HSV genes including the HSV polymerase UL30, the polymerase accessory factor UL42, and the origin-binding protein UL9.

In certain embodiments, the recombinant HSV vector of the present invention has, in addition to the ICP27 deletion described herein, deletion of one or more further immediate-early (IE) genes encoding infected cell proteins (ICPs), such as ICP0, ICP4, ICP22, and/or ICP47. The production of the subject replication-incompetent rHSV vectors requires adequate complementing cell lines for providing in trans the missing replication and packaging functions of rHSV.

In certain embodiments, in addition to ICP27 deletion, the subject rHSV further lacks gene(s) encoding HSV glycoprotein H (gH). Infectious particles could be generated from such rHSV only from a complementing gH-expressing cell line, thus conferring a further level of safety.

Other than the essential feature relating to the ICP27 deletion described herein, the subject rHSV vector may retain most of the wild-type HSV genome, or have more than 50% of the wild-type HSV genome encoding nonessential gene products deleted without jeopardizing viral amplification. The subject rHSV vector may also comprise two cis-acting elements required for HSV replication and packaging—the origin of replication (oriS), and the packaging signal (sequence a or pac).

In certain embodiments, the subject rHSV and/or rAAV vectors are produced in in vitro culture conditions, such as in bioreactors (e.g., 0.5L, 1L, 2L, 3L, 5L, 10L, 20L, 50L, 100L, 250L, 500L, or 1,000L working volume bioreactors), such as a CelliGen Plus packed-bed bioreactor (New Brunswick Scientific) for fed-batch vector production for 3 days post infection.

In certain embodiments, the rHSV vectors of the present invention are produced as in vitro culture on adherence-dependent cell lines, such as Vero and Vero-derived cell lines, that rely on a solid support. In certain embodiments, the solid support is a tissue culture surface, such as tissue culture dishes, plates, bottles, flasks, cell factory, etc. In certain embodiments, the solid support is a microcarrier, such as Cytodex 1 (GE Healthcare Life Sciences, Piscataway, NJ); a macrocarrier, such as FibraCel (New Brunswick Scientific, Edison, NJ), or a multilayered culture vessel, such as a CellCube (Corning Life Sciences, Lowell, MA) that permit medium perfusion.

In certain embodiments, the subject rHSV and/or rAAV vectors are produced as in vitro culture in eukaryote cells adapted to grow in suspension, such as a suspension culture of the BHK cell line, such as a BHK cell that express an ICP27 coding sequence of the invention that does not overlap with the ICP27 deletion on the rHSV vector.

In certain embodiments, the culture supernatant yields in excess of 1×10¹⁰ plaque-forming units (PFU) of rHSV, 1×10¹¹ plaque-forming units (PFU) of rHSV, 1×10¹² plaque-forming units (PFU) of rHSV, 1×10¹³ plaque-forming units (PFU) of rHSV, 1×10¹⁴ plaque-forming units (PFU) of rHSV.

In certain embodiments, vector stock is produced by one or more post processing steps, such as filtration and/or concentration (e.g., depth filtration, dead-end filtration, tangential flow filtration (TFF), and diafiltration), multi-column chromatography purification, final concentration/buffer exchange, etc., to obtain vector stocks with sufficient purity for administration to animals, including human. In certain embodiments, the purification process and the purified vector stock satisfy GMP standard.

In certain embodiments, the titer of the rHSV and/or rAAV vector stocks is about 1-2×10⁷ PFU/ml, about 1-2×10⁸ PFU/ml, about 1-2×10⁹ PFU/ml, about 1-2×10¹⁰ PFU/ml, about 1-2×10¹¹ PFU/ml, or about 1-2×10¹² PFU/ml.

In certain embodiments, the total yield of the rAAV vector stock is about 1-25×10¹⁴ total VG of purified rAAV, about 1-10×10¹⁴ total VG of purified rAAV, about 1-5×10¹⁴ total VG of purified rAAV, or about 2-4×10¹⁴ total VG of purified rAAV.

In certain embodiments, rHSV and/or rAAV vectors so produced are further purified from crude cell lysates by ion-exchange chromatography and/or by iodixanol density gradient centrifugation to ensure high final product purity. In certain embodiments, rAAV vectors so produced qualify as a clinical-grade vector batch.

In certain embodiments, the AAV production method of the invention further comprises determining the titer, purity, and/or potency of the rAAV vectors so produced. This may include characterizing the purified rAAV stocks using one or more of: silver staining of SDS-PAGE separation of proteins to determine purity, qPCR to determine the ratio of rAAV full capsids to infectious particles (TCID₅₀), ELISA to determine the residual HSV protein, and qPCR to determine the residual HSV DNA.

### 7. Treatment of Muscular Dystrophy Using AAV Produced by the rHSV

The subject rHSV-based system can be used for large scale production of rAAV, which in turn can be used in gene therapy for treating various forms of muscular dystrophy, such as Duchenne's muscular dystrophy (DMD), Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), etc. In certain embodiments, the muscular dystrophy is DMD or BMD.

Thus another aspect of the invention provides a method of treating muscular dystrophy (such as DMD and BMD) in a subject in need thereof, the method comprising administering to the subject a therapeutically effective amount of a recombinant AAV (rAAV) vector encoding a functional version of the gene defective in the muscular dystrophy, such as a microdystrophin gene, wherein the rAAV is produced by the method of the invention using the subject rHSV vector and complementary system.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272 (all incorporated herein by reference).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the method further comprises producing the rAAV by the method of the invention using the subject rHSV vector and complementary system, prior to administering to the subject the rAAV so produced.

### EXAMPLES

### Example 1 Generation of All-In-One (A-1-0) rHSV Vectors with Complete ICP27 Deletion and Insertion into the UL43 Locus with Both a Human Dystrophin Minigene and an AAV Rep/Cap Coding Sequence

To produce exemplary A-I-O HSV vectors of the invention, the ICP27/UL54 locus was completely deleted, followed by insertion of both a gene-of-interest (GOI) flanked by AAV ITR sequences, and an AAV Rep/Cap coding sequence into the UL43 locus.

Specifically, ICP27 gene (UL54), including its promoter and coding sequence, was first completely deleted from the wild-type HSV-1 KOS 1.1 strain genome integrated in a bacterial artificial chromosome (BAC) vector (HSV-1 KOS 1.1-BAC), by using homologous recombination in electrocompetent E. coli. Four of the independently isolated ICP27-deleted HSV-1-BAC clones with 2.1-kb (2,077 bp) deletion in UL54 gene were tested using the V27 and Vero cell plaque assay to show that robust production of ICP27-deleted HSV-1 virus can be achieved from these ICP27-deleted HSV-1-BAC clones with essentially no rcHSV contamination (data not shown). SEQ ID NO: 1 is a polypeptide sequence of ICP27 HSV-1 strain KOS 1.1 ICP27 peptide (SEQ ID NO: 1; GenPept Accession AAF43147).

The homologous recombination in electrocompetent E. coli technology is based on homologous recombination and about 50 bp of homology regions on each side flanking a target DNA sequence. This is a system that can modify a target DNA, such as the wild-type HSV genome on a BAC vector, in particular the ICP27 gene, by deleting the target DNA.

One such electrocompetent E. coli strain is DY380, which is derived from the DH10B E. coli strain. Another such strain is SW102, which is derived from DY380. The galactose operon in SW 102 has been modified, such that it is fully functional, except that the galactokinase gene (galK) has been deleted, but the galK function can be added in trans, thereby restoring the ability to grow on galactose as carbon source. This forms the biological basis for galK based selection in SW102.

The galK based selection is a two-step system involving both positive selection and negative selection. First, during the positive selection step, a galK cassette containing homology (e.g., at least 50 bp on each side) to a specified position in a BAC, such as the ICP27 gene locus, is inserted by homologous recombination into the BAC. The resulting recombinant bacteria are then able to grow on minimal media with galactose as the sole carbon source (positive selection). Second, the galK cassette is substituted by a donor sequence with homology flanking the galK cassette in the BAC vector. Successful recombinants can be identified by selecting against the galK cassette based on resistance to 2-deoxy-galactose (DOG) on minimal plates with glycerol as the carbon source. Although DOG itself is harmless, galK can phosphorylate DOG to become 2-deoxy-galactose-1-phosphate, a non-metabolizable and therefore toxic intermediate to the bacteria host. Thus only bacteria that have lost the galK cassette (e.g., by recombination) will survive and become DOG-resistant colonies (negative selection).

Using this system, a BAC carrying the wild-type HSV-1 KOS 1.1 strain was first introduced into the E. coli SW 102 strain by electroporation. Next, a galK cassette with the galK coding sequence flanked on each side by about 50 bp of sequences homologous to genomic regions flanking the ICP27 gene was generated by PCR amplification. Here, the 50 bp homologous regions flanking the ICP27 gene were designed to eliminate the ICP20 gene completely, which is the 2,077 bp of HSV-1 KOS 1.1 DNA sequence of the UL54 gene (GenBank Accession KT887224; nts: 113,013-115,089). See SEQ ID NO: 2. This deletion includes the 538-bp UL54 promoter (nucleotides 1-538 of SEQ ID NO: 2), and the 1539-bp ICP27 coding sequence (nucleotides 539-2077 of SEQ ID NO: 2).

Following homologous recombination, galK positive selection was performed to identify recombinants that had replaced the 2,077-bp ICP27 gene with the galK cassette.

Next, galK was similarly eliminated from the BAC vector using homologous recombination by using ICP27 flanking sequences (linking about 50 bp upstream of nucleotide 113,013 in KT887224, to about 50 bp downstream of nucleotide 115,089 in KT887224). Following this step, galK negative selection was performed to identify recombinants that had lost the galK cassette. The resulting BAC clones were named ICP27-deleted HSV-1-BAC clones. Four of such clones were subject to further testing to show their ability to support robust rHSV production with essentially no contaminating rcHSV.

Specifically, BHK153 cells were transfected by BAC DNA from individual ICP27-deleted HSV-1-BAC clones. The BAC DNA sequence from ICP27-deleted HSV-1-BAC clones share no homologous region with the ICP27 gene stably integrated in the BHK153 cells, which have previously been confirmed to be able to provide ICP27 function for rHSV production. Lysates of the transfected BHK153 cells were collected about 12-13 days post transfection, and supernatants containing rHSV were assayed in 6-well plates, to determine rHSV viral titer on V27 cells and Vero cells.

The ICP27-deleted HSV-1-BAC vector was then further modified by homologous recombination to insert into the HSV-1 UL43 locus, with both a gene of interest (GOI), such as a human dystrophin minigene (microdystrophin), and an AAV rep/cap expression cassette required for rAAV production.

One way to accomplish this is by using a two-step process using galK selection as described above. First, the UL43 locus is inserted by a galK cassette via electroporation and recombination. The galK cassette is amplified by PCR using two primers, each primer flanks the galK cassette and amplifies sequences of at least a 50-bp sequence on each side of the insertion site within the HSV-1 UL43 locus sequence. The resulting PCR product has the galK coding sequence, flanked by two 50-bp UL43 homologous regions for electroporation and recombination. The resulting PCR product is then introduced into E. coli SW 102 having ICP27-deleted HSV-1-BAC clones to perform homologous recombination. Positive galK selection resulted in a modified ICP27-deleted HSV-1-BAC genome, having both complete ICP27 deletion and a galK gene insertion into HSV UL43 locus (ICP27-deleted HSV-1-BAC UL43^{mut}/galK⁺).

In the next step, galK is replaced with a GOI and rep/cap DNA cassettes via electroporation and recombination, resulting in the A-I-O rHSV vector for rAAV/HSV production.

This process can be used to produce A-I-O HSV vectors comprising any GOI to be packaged into AAV viral particles. One of such gene of interest may be a dystrophin minigene described in PCT/US2016/013733 (published as WO/2016/115543, incorporated herein by reference). One such specific microdystrophin gene comprises a coding sequence for the R1, R16, R17, R23, and R24 spectrin-like repeats of the full-length dystrophin protein, under the transcriptional control of the CK8 promoter, and is referred to herein as “CK8-HuDys5.”

In the above constructs, the HSV genome was inserted in a pBAC plasmid, and that inserted sequence was flanked by loxP sequences. The BAC sequence can be removed from the ICP27-deleted HSV-1-BAC UL43^{mut}/ITR-GOI-ITR-repcap backbone, by co-transfecting the BAC vector and a Cre-expressing plasmid into ICP27 expressing BHK or Vero cells. Plaque purification of the progeny viruses lacking BAC sequence results in virus ICP27-deleted HSV-1 UL43^{mut}/ITR-GOI-ITR-repcap.

Recombinant AAV vectors can be produced using this rHSV vector. The resulting rAAV vectors have within the ITR sequences a human dystrophin minigene HuDys5 under the control of the muscle specific CK8 promoter, and can be readily used in gene therapy to treat muscular dystrophy.

A slight variation of the process above may include directly replacing any GOI by any other GOI using homologous recombination. For example, after the generation of the ICP27-deleted HSV-1 UL43^{mut}/ITR-GOI-ITR-repcap clone, the GOI may be directly replaced by another GOI, via galK selection. For example, if the promoter and the poly A regions of the old and new GOI's are the same, they can serve as the 50-bp minimal homologous regions for homologous recombination. If these sequences are different, then the spacer regions can be used (if they are at least 50 bp in length).

In particular, assuming the promoter and polyA regions are identical, the galK cassette can first be inserted into ICP27-deleted HSV-1 UL43^{mut}/ITR-GOI-ITR-repcap between the promoter and polyA sequences of the GOI coding sequence by electroporation and recombination. Following galK positive selection, ICP27-deleted HSV-1 UL43^{mut}/ITR-promoter-galK-polyA-ITR is obtained in which the coding sequence of GOI, between its promoter and the polyA signal sequence, is replaced by galK. Next, the inserted galK cassette in ICP27-deleted HSV-1 UL43^{mut}/ITR-prom-galK-polyA-ITR-repcap is replaced by the coding sequence from another GOI, via electroporation and recombination. ICP27-deleted HSV-1 UL43^{mut}/ITR-GOI-ITR-repcap is obtained following galK negative selection.

The BAC sequence can be removed from the ICP27-deleted HSV-1-BAC UL43^{mut}/ITR-GOI-ITR-repcap, by transfecting the BAC vector and a Cre-expressing plasmid into ICP27 expressing BHK or Vero cells. Plaque purification of the progeny viruses lacking BAC sequence results in virus ICP27-deleted HSV-1 UL43^{mut}/ITR-GOI-ITR-repcapX.

### Example 2 Generation of All-In-One (A-1-0) rHSV Vectors with Complete ICP27 Deletion and Insertion into the ULA3 Locus with Both a Human Dystrophin Minigene and an AAV Rep/Cap Coding Sequence—FRT Constructs

In this example, and similar to Example 1, the ICP27-deleted HSV-1-BAC vector in Example 1 is further modified by inserting into the HSV-1 UL43 gene locus both a gene of interest, such as a human dystrophin minigene, and an AAV rep/cap expression cassette required for rAAV production. The difference between this example and Example 1 is that the galK cassette in the intermediate construct, as well as the GOI (for example the dystrophin minigene) and the rep-cap expression cassette, are all flanked by frtG and frtH sequences to facilitate easier exchange of constructs. Thus electroporation and recombination in E. coli is only used initially to replace the UL43 locus with the galK cassette flanked by frtG and frtH sites, and galK can then be replaced by the GOI and the rep-cap expression cassette via FLP recombination.

This is again accomplished in a two-step process using galK selection as described above. First, the UL43 locus is replaced by a frtG-galK-frtH cassette via electroporation and recombination. The frtG-galK-frtH cassette is amplified by PCR with two primers, each having a 50-bp sequence homologous to a sequence flanking the HSV-1 UL43 locus. The resulting PCR product has the galK coding sequence in the middle, flanked by frtG and frtH, which are in turn flanked by two 50-bp UL43 locus homologous regions for homologous recombination. The PCR product is then introduced into E. coli (for example, SW102) having the ICP27-deleted HSV-1-BAC clones (see Example 1) for homologous recombination. Positive galK selection results in a modified rHSV on BAC vector, having both complete ICP27 deletion and UL43 deletion/insertion, and having galK flanked by frtG and frtH sites (ICP27-deleted HSV-1-BAC UL43^{mut}/frtG-galK-frtH). This BAC clone is then used subsequently as acceptor for any ITR-GOI-ITR cassettes, and any rep-cap cassettes, via FLP recombination.

In the next step, galK is replaced with the GOI-repcap construct via FLP recombination, resulting in an A-I-O rHSV vector useful for rAAV production.

One of such rHSV vectors has a gene of interest (GOI) flanked by the AAV ITR sequences, and the gene of interest may be a dystrophin minigene described in PCT/US2016/013733 (published as WO/2016/115543, incorporated herein by reference). One such specific microdystrophin gene comprises a coding sequence for the R1, R16, R17, R23, and R24 spectrin-like repeats of the full-length dystrophin protein, under the transcriptional control of the CK8 promoter, and is referred to herein as “CK8-HuDys5.” Alternatively, any of a different dystrophin minigene, such as those described herein above, may be inserted.

The gene of interest flanked by the AAV ITR sequences, which is adjacent to the rep-cap expression cassette, are further flanked by the frtG and frtH sites (frtG-ITR-GOI-ITR-repcap-frtH). The entire construct can be carried on a transfer plasmid for FLP recombination on ICP27-deleted HSV-1-BAC UL43^{mut}/frtG-galK-frtH. After galK negative selection, successful recombinants having lost galK can be obtained as ICP27-deleted HSV-1-BAC UL43^{mut}/frtG-ITR-GOI-ITR-repcap-frtH, a modified rHSV vector on the BAC vector, comprising a completely deleted ICP27 (UL54) gene and a mutant UL43, the latter of which is replaced with/inserted into by a GOI flanked by AAV ITR sequences and a repcap expression cassette, both flanked by frtG-frtH sites.

The GOI and the repcap expression cassette can be inserted together, or one after another (whichever is inserted first), with minor modification of the procedure described above.

The loxP-flanked BAC backbone in ICP27-deleted HSV-1-BAC UL43^{mut}/frtG-ITR-GOI-ITR-repcap-frtH can be eliminated by co-transfecting the BAC vector and a Cre-expressing plasmid into Vero cells. Plaque purification of the progeny viruses lacking beta galactosidase results in virus ICP27-deleted HSV-1-BAC UL43^{mut}/frtG-ITR-GOI-ITR-repcapX-frtH.

rAAV vectors can be produced using the subject A-I-O rHSV vector, by infecting a suitable production cell line having the required helper virus proteins. The resulting rAAV vectors have within the ITR sequences any GOI, such as a human dystrophin minigene, and can be readily used in gene therapy to treat muscular dystrophy.

### Example 3 Generation of V75.4 Cell Line

This example shows the establishment of a packaging cell line (e.g., V75.4 cell line, or V75) for producing HSV vectors having deleted ICP27 gene. The packaging cell line contains an ICP27-encoding sequence that is designed to have no sequence overlap with the HSV vector of the invention with a complete deletion of the ICP27 gene.

The parental cell line used for the generation of V75.4 Cell Line was a Vero cell line CCL-81, obtained from American Type Culture Collection (ATCC) (Manassas, VA). Vero cell line is an African green monkey (Cercopithecus aethiops) kidney cell line which is highly susceptible to various types of viruses, including herpes simplex virus 1 (HSV-1). Vero cells were stably transduced by a lentivirus vector generated from a lenti-provirus plasmid, by inserting the nucleotide sequence UL54-002 (promoter and codon-optimized ORF) SEQ ID NO: 3. The ICP27 expression cassette stably integrated in the V75.4 cells contains woodchuck hepatitis virus post-transcriptional regulatory element (WPRE) sequence and the lentivirus polyadenylation signal from 3′ LTR lentivirus sequence, as is shown in SEQ ID NO: 4.

### Transduction, Cloning and Cell Line Identification

Vero cells maintained in DMEM plus 5% FBS at 37° C. in a humidified CO₂-controlled incubator with 5% CO₂ were transduced with lentivirus vector PR-UL54-002, at multiplicity of infection (MOI)=10. Cells from this pool were cloned by plating in different densities into 15 cm² plates, and were maintained in DMEM plus 10% FBS without selection at 37° C. in a humidified CO₂-controlled incubator with 5% CO₂. After 2-3 weeks, the single colonies-master wells (MWs) were detached by trypsin and harvested using cloning cylinders. Cells from master wells (MWs) were seeded into 24-well plates and maintained in the same medium and conditions. As the wells reached confluence, clones were detached using trypsin and expanded to two 24-well plates (one for terminal replica for screening and one for expansion). The portion of the culture slated for HSV production screening was infected with Δ27HSV at MOI=0.15 and harvested after 72 hrs and tested for DDPCR titer for HSV UL36 copies and plaque per mL.

Four outperforming MWs were identified and shown in Table 1.

TABLE 1
Outperforming MWs ddPCR (UL36) and plaque titers per mL

| MW CLONE | ddPCR TITER (VG/mL) | V27 TITER (PFU/mL) |
|---|---|---|
| LV-Vero002 #28 | 3.61E+08 | 2.00E+06 |
| LV-Vero002 #36 | 4.11E+08 | 1.20E+07 |
| LV-Vero002 #75 | 5.49E+08 | 2.20E+07 |
| LV-Vero002 #76 | 4.07E+08 | 8.00E+06 |

Ultimately the highest level of production was found in MW LV-Vero002 #75 (MW75) that had been selected for further subcloning.

Cells from the MW75 were cloned by limiting dilution seeded at a density of 0.3 cells/well in 96-well plates in the same medium and conditions as described previously. The plates were visually inspected to identify those wells seeded with only a single cell. After a week of growth, the media was replaced and in the wells that reached confluence, clones were detached using trypsin and expanded to 24 well plates. As the wells reached confluence, clones were detached using trypsin and expanded to two 24-well plates (one for terminal replica for screening and one for expansion). The portion of the culture slated for HSV production screening was infected with Δ27HSV at MOI=0.15 and harvested after 72 hrs and tested for ddPCR titer for HSV UL36 copies. The outperforming MW75 subclones were selected by UL36 ddPCR.

The subclones were narrowed down to clones #4, #20 and #24, and were assessed for the viability, coupling time, and generational stability up to passage P26. The stability and the yield of Δ27HSV virus was the best in MW75 clone #4 (hence the V75.4 cell line), which was selected for further use in the rc-HSV-free system of the invention.

The ICP-27 coding sequence in these newly established packaging cell lines, including V75.4, contains a codon optimized region encoding the C-terminal end of the ICP27 protein. The codon optimization was designed to have least sequence homology, at the nucleic acid level, compared to the remnant ICP27 coding sequence in the d27-1 HSV vector currently wildly used. This was designed to minimize the chance of generating rcHSV viral particles, when the traditional d27-1 HSV vector is packaged in the subject V75.4-type packaging cell lines, since any sequence overlap between the remnant ICP27 coding sequence on the d27-1 HSV vector and the ICP27 encoding sequence in the subject packaging cell line will be reduced to 67% or less due to codon degeneracy.

Indeed, preliminary results (data not shown) indicated that no rcHSV viral particles were generated when the traditional d27-1 HSV vector was packaged in V75.4 cell line, to the extent that there was no contaminating rcHSV in the d27-1 HSV stock.

### Example 4 A-1-O UL43 HSV Vectors Minimized DNA Mis-Packaging in AAV

This example demonstrates a surprising finding of greatly reduced AAV mis-packaging when the subject A-I-O HSV vectors were used to produce AAV in Expi293 cells.

In the traditional dual HSV vector-based AAV production system, two separate HSV vectors are required—one carries an GOI flanked by AAV ITR sequences, and the other carries a rep-cap expression cassette that produces the required AAV Rep and Cap proteins for packaging. Both HSV vectors are used to co-infect a suitable AAV producing cell line, such as the Expi293F cells (which does not express ICP27 required for HSV replication). In the presence of the helper virus proteins (such as those supplied by Adenovirus), AAV packaging is enabled within the producing cell line, and the GOI flanked by the AAV ITR sequences are packaged into the AAV viral capsids expressed from the rep-cap expression cassette carried on by the other HSV vector.

It was found that, when the repcap cassette and the GOI cassette were both inserted into the UL43 locus, occasionally and at relatively low frequency (e.g., 1-2%), certain HSV vector sequences outside the ITR sequences are mis-packaged into the same AAV viral particles, resulting in mis-packaged AAV viral packages having non-productive HSV vector sequences.

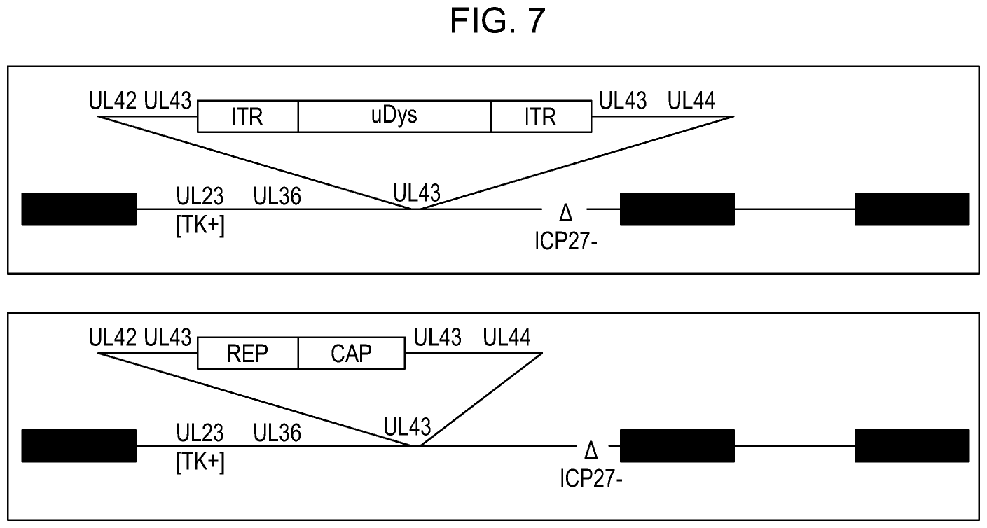

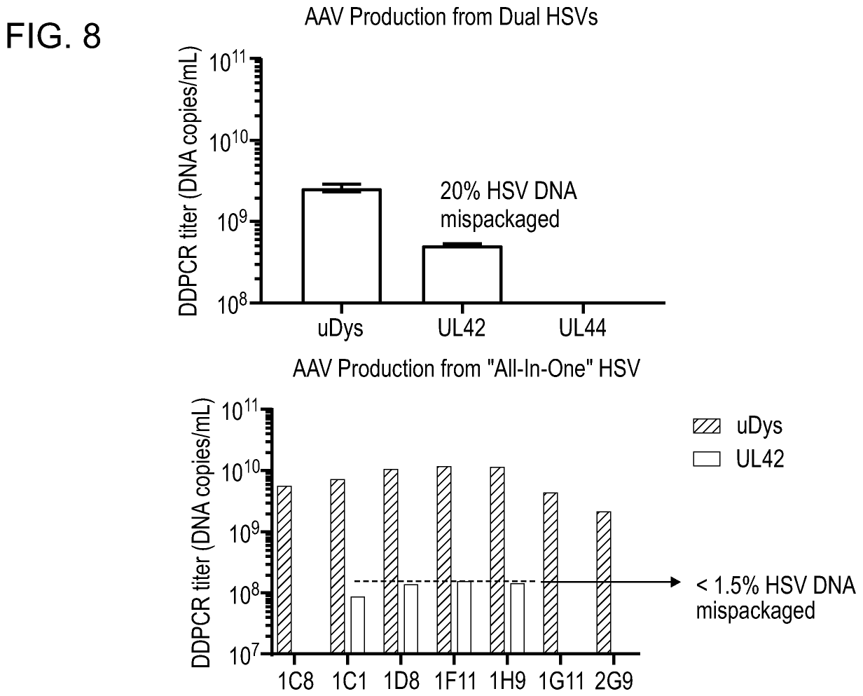

For example, FIG. 7 shows schematic drawings of such a pair of HSV vectors of the traditional dual HSV vector system. FIG. 8 shows mis-packaging of AAV produced from a dual HSV system happens at a frequency of generally about 8-20% compared to the correctly packaged GOI. The mis-packaging appears uni-directional, with only HSV vector DNA found at one (but not the other) side/end of the ITR-flanked GOI found within the mis-packaged AAV. The mis-packaging also appeared to have limited reach, in that only HSV DNA adjacent to (but not far away from) the insertion site (e.g., near one of the ITR sequences) was found to be mis-packaged. See U124 titers in the table below, compared to the corresponding Ul42 titers.

Thus in the one GOI construct inserted into the UL43 locus (d43RC9&GOI), only the UL42 HSV sequence was detected in mis-packaged AAV, while UL44 (on the other end of the insertion) and UL24 (farther away from the insertion point) sequences were undetectable.

Surprisingly, when the subject A-I-O HSV vector was used to produce AAV directly in the same cell line (Expi293F), at the presence of the required helper, rate of AAV mis-packaging was greatly reduced. In 4 of the 7 test clones, the highest rate of mis-packaging is less than 1.5%, a 14-15 fold reduction compared to the dual vector system rate. Indeed, three of the tested clones produced no detectable level of mis-packaged AAV (FIG. 8).

Representative experimental results are summarized in the table below. Here, the measured Ul42 titers represented mis-packaging. Compared to the corresponding AAV titers, the percentage of mis-packaging ranged between a low of 0.39% to a high of only 0.86%. These represent a dramatic drop in mis-packaging rate of up to about 20% in FIG. 8.

AAV Production in AIO

| Expi293 | Ul36 titer | AAV Titer |
|---|---|---|
| MOI 5.7 | 2.08E+07 | 1.00E+11 |
| MOI 11.5 | 2.95E+07 | 4.83E+10 |

| | Ul36 titer | AAV Titer | Ul24 Titer | Ul42 Titer | Ul42/uDYS |
|---|---|---|---|---|---|
| MOI 1 | 1.24E+07 | 4.79E+10 | 1.22E+07 | 4.14E+08 | 0.86% |
| MOI 2 | 1.40E+07 | 6.12E+10 | 1.46E+07 | 4.28E+08 | 0.70% |
| MOI 4 | 8.82E+06 | 5.92E+10 | 1.65E+07 | 2.31E+08 | 0.39% |

### Example 5 Higher Titer of AAV Prepared Using the A-1-O HSV Vectors in ICP27-Expressing V75 Cells Vs. ICP27 Negative HEK293 Cells

This example shows that the subject A-I-O HSV vectors surprisingly produced higher AAV titers in the ICP-27-expressing V75 cells, compared to that in the ICP-27 negative HEK293 cells.

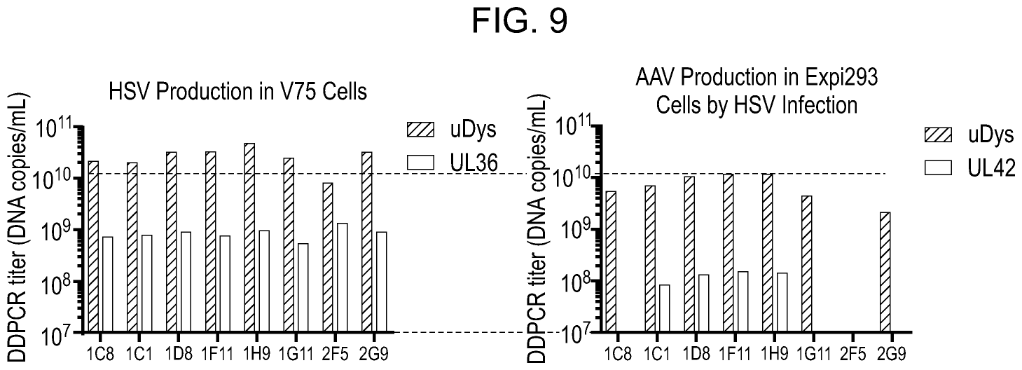

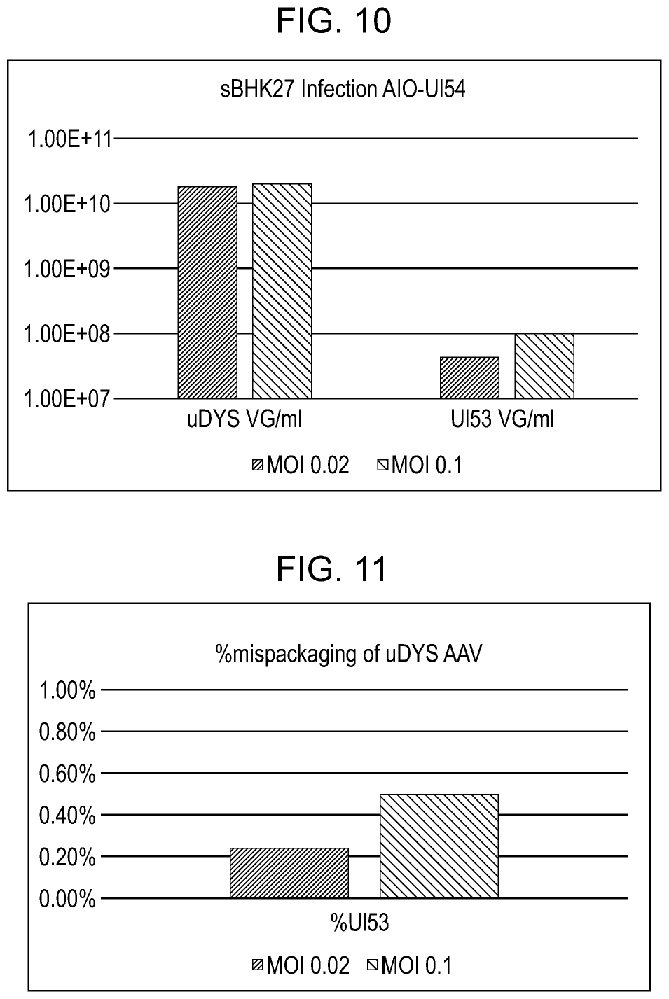

Specifically, eight different clones of A-I-O HSV vectors of the invention were used to produce AAV, either in the ICP-27-expressing V75 cells, or in the ICP-27 negative Expi293F cells. The titers of the resulting AAV stocks were determined and directly compared in pairs. FIG. 9 shows that, in each direct comparison, V75 cells consistently produced 100-300% more (up to 10-fold more in 2G9) AAV than Expi293 cells.

Further, the AAV viral stocks produced by the A-I-O vector system of the invention demonstrated very high potency or expression level, equivalent to that of the AAV viral stocks produced by the conventional triple transfection methods.

Specifically, in one experiment, mouse differentiated myoblast C2C12 cells were transduced at low multiplicity of infections (MOIs) with microdystrophin-expressing A-I-O vectors packaged in AAV9 capsid, produced using either the sBHK27 cells grown in suspension (A-I-O AAV #153), or the V75 cells grown as adherent cells (A-I-O AAV #154). As control, AAV stocks produced by infection with dual HSV system in suspension 293 cells coinfected with both HSV-goi and HSVrepcap—AAV-Dual #1 and AAV-Dual #2—were also used in the same experiment. AAV-Dual #2 was also used as the reference (100%) to get the relative potency of the other AAV samples in the assay. Cells were harvested 96 hours after transduction, and microdystrophin expression was measured.

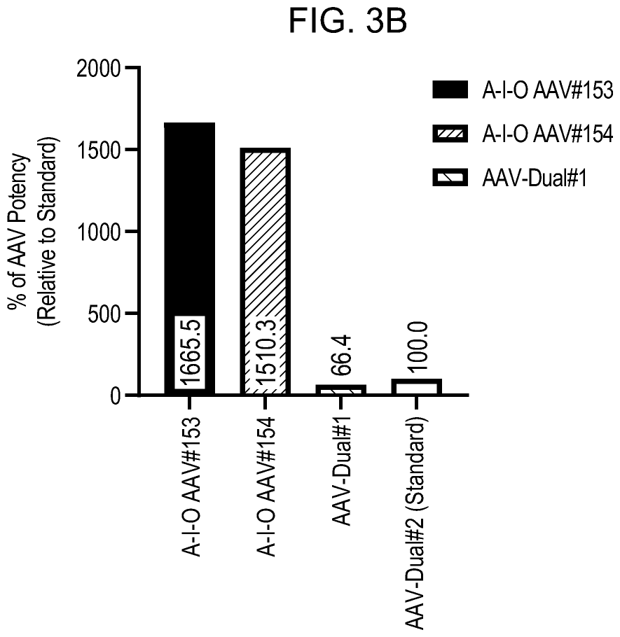

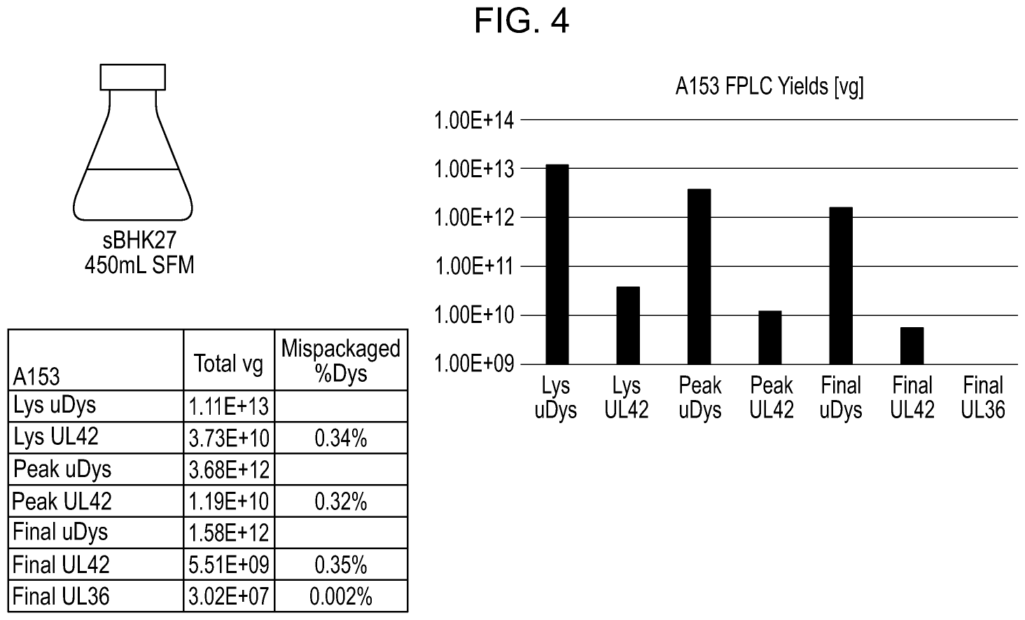

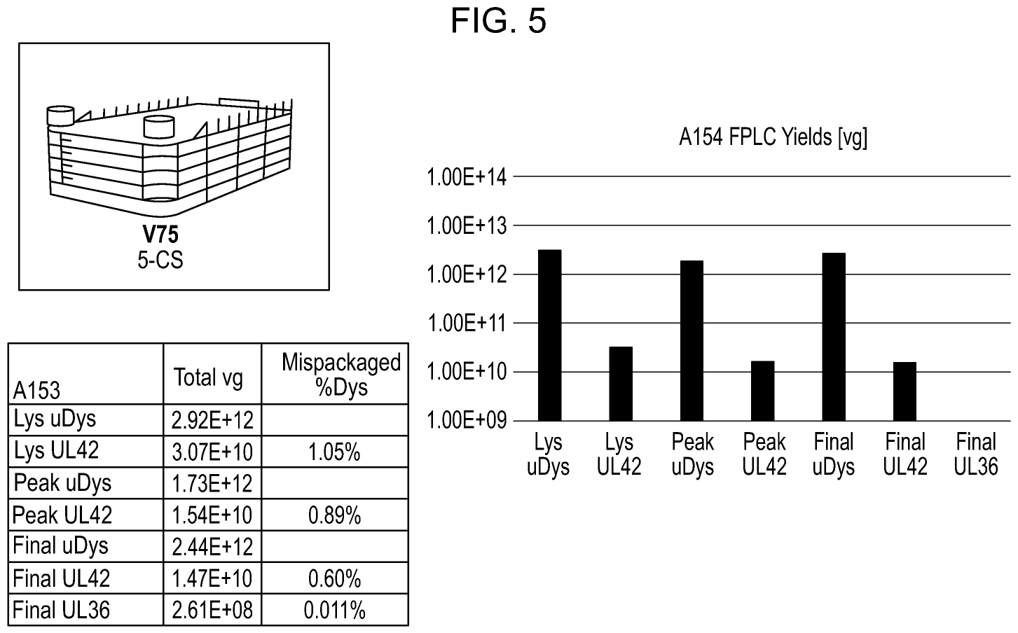

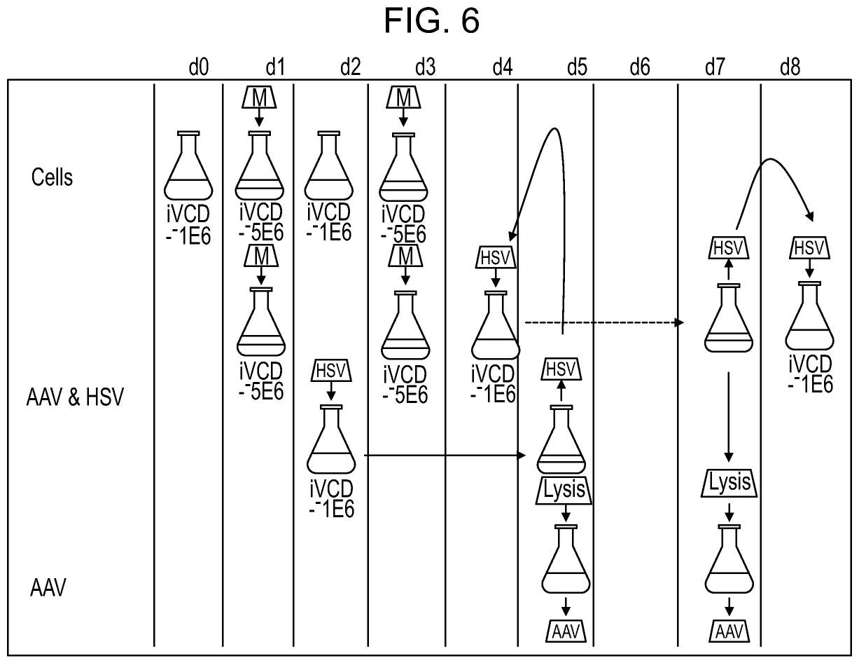

The All-In-One (A-I-O) HSV produced AAV samples, A-I-O AAV #153 and A-I-O AAV #154 were found to have 23-25 fold- or 15-16 fold-higher microdystrophin expression than the dual HSV system-produced AAV Lots (i.e., AAV-Dual #1 or AAV-Dual #2), respectively. See FIG. 3B.

DNA AND PEPTIDE SEQUENCES
(SEQ ID NO: 1)
MATDIDMLIDLGLDLSDSDLDEDPPEPAESRRDDLESDSNGECSSSDEDMEDPHGEDGPEPILDAARP
AVRPSRPEDPGVPSTQTPRPTERQGPNDPQPAPHSVWSRLGARRPSCSPERHGGKVARLQPPPTKAQP
ARGGRRGRRRGRGRGGPGAADGLSDPRRRAPRTNRNPGGPRPGAGWTDGPGAPHGEAWRGSEQPDPPG
GPRTRSVRQAPPPLMTLAIAPPPADPRAPAPERKAPAADTIDATTRLVLRSISERAAVDRISESFGRS
AQVMHDPFGGQPFPAANSPWAPVLAGQGGPFDAETRRVSWETLVAHGPSLYRTFAGNPRAASTAKAMR
DCVLRQENFIEALASADETLAWCKMCIHHNLPLRPQDPIIGTAAAVLDNLATRLRPFLQCYLKARGLC
GLDELCSRRRLADIKDIASFVFVILARLANRVERGVAEIDYATLGVGVGEKMHFYLPGACMAGLIEIL
DTHRQECSSRVCELTASHIVAPPYVHGKYFYCNSLF*
(SEQ ID NO: 2)
CGTCACATCCAGGCCGGCGGAAACCGGAACGGCATATGCAAACTGGAAACTGTCCTGTCTTGGGGCCC
ACCCACCCGACGCGTCATATGTAAATGAAAATCGTTCCCCCGAGGCCATGTGTAGCCTGGATCCCAAC
GACCCCGCCCATGGGTCCCAATTGGCCGTCCCGTTACCAAGACCAACCCAGCCAGCGTATCCACCCCC
GCCCGGGTCCCCGCGGAAGCGGAACGGTGTATGTGATATGCTAATTAAATACATGCCACGTACTTATG
GTGTCTGATTGGTCCTTGTCTGTGCCGGAGGTGGGGCGGGGGCCCCGCCCGGGGGGCGGAACTAGGAG
GGGTTTGGGAGAGCCGGCCCCGGCACCACGGGTATAAGGACATCCACCACCCGGCCGGTGGTGGTGTG
CAGCCGTGTTCCAACCACGGTCACGCTTCGGTGCCTCTCCCCGATTCGGGCCCGGTCGCTTGCTACCG
GTGCGCCACCACCAGAGGCCATATCCGACACCCCAGCCCCGACGGCAGCCGACAGCCCGGTCATGGCG
ACTGACATTGATATGCTAATTGACCTCGGCCTGGACCTCTCCGACAGCGATCTGGACGAGGACCCCCC
CGAGCCGGCGGAGAGCCGCCGCGACGACCTGGAATCGGACAGCAACGGGGAGTGTTCCTCGTCGGACG
AGGACATGGAAGACCCCCACGGAGAGGACGGACCGGAGCCGATACTCGACGCCGCTCGCCCGGCGGTC
CGCCCGTCTCGTCCAGAAGACCCCGGCGTACCCAGCACCCAGACGCCTCGTCCGACGGAGCGGCAGGG
CCCCAACGATCCTCAACCAGCGCCCCACAGTGTGTGGTCGCGCCTCGGGGCCCGGCGACCGTCTTGCT
CCCCCGAGCGGCACGGGGGCAAGGTGGCCCGCCTCCAACCCCCACCGACCAAAGCCCAGCCTGCCCGC
GGCGGACGCCGTGGGCGTCGCAGGGGTCGGGGTCGCGGTGGTCCCGGGGCCGCCGATGGTTTGTCGGA
CCCCCGCCGGCGTGCCCCCAGAACCAATCGCAACCCGGGGGGACCCCGCCCCGGGGGGGGGTGGACGG
ACGGCCCCGGCGCCCCCCATGGCGAGGCGTGGCGCGGAAGTGAGCAGCCCGACCCACCCGGAGGCCCG
CGGACACGGAGCGTGCGCCAAGCACCCCCCCCGCTAATGACGCTGGCGATTGCCCCCCCGCCCGCGGA
CCCCCGCGCCCCGGCCCCGGAGCGAAAGGCGCCCGCCGCCGACACCATCGACGCCACCACGCGGTTGG
TCCTGCGCTCCATCTCCGAGCGCGCGGCGGTCGACCGCATCAGCGAGAGCTTCGGCCGCAGCGCACAG
GTCATGCACGACCCCTTTGGGGGGCAGCCGTTTCCCGCCGCGAATAGCCCCTGGGCCCCGGTGCTGGC
GGGCCAAGGAGGGCCCTTTGACGCCGAGACCAGACGGGTCTCCTGGGAAACCTTGGTCGCCCACGGCC
CGAGCCTCTATCGCACTTTTGCCGGCAATCCTCGGGCCGCATCGACCGCCAAGGCCATGCGCGACTGC
GTGCTGCGCCAAGAAAATTTCATCGAGGCGCTGGCCTCCGCCGACGAGACGCTGGCGTGGTGCAAGAT
GTGCATCCACCACAACCTGCCGCTGCGCCCCCAGGACCCCATTATCGGGACGGCCGCGGCGGTGCTGG
ATAACCTCGCCACGCGCCTGCGGCCCTTTCTCCAGTGCTACCTGAAGGCGCGAGGCCTGTGCGGCCTG
GACGAACTGTGTTCGCGGCGGCGTCTGGCGGACATTAAGGACATTGCATCCTTCGTGTTTGTCATTCT
GGCCAGGCTCGCCAACCGCGTCGAGCGTGGCGTCGCGGAGATCGACTACGCGACCCTTGGTGTCGGGG
TCGGAGAGAAGATGCATTTCTACCTCCCCGGGGCCTGCATGGCGGGCCTGATCGAAATCCTAGACACG
CACCGCCAGGAGTGTTCGAGTCGTGTCTGCGAGTTGACGGCCAGTCACATCGTCGCCCCCCCGTACGT
GCACGGCAAATATTTTTATTGCAACTCCCTGTTTTAG
(SEQ ID NO: 3)
CCACCCCCGCCCGGGTCCCCGCGGAAGCGGAACGGTGTATGTGATATGCTAATTAAATACATGCCACG
TACTTATGGTGTCTGATTGGTCCTTGTCTGTGCCGGAGGTGGGGCGGGGGCCCCGCCCGGGGGGCGGA
ACTAGGAGGGGTTTGGGAGAGCCGGCCCCGGCACCACGGGTATAAGGACATCCACCACCCGGCCGGTG
GTGGTGTGCAGCCGTGTTCCAACCACGGTCACGCTTCGGTGCCTCTCCCCGATTCGGGCCCGGTCGCT
TGCTACCGGTGCGCCACCACCAGAGGCCATATCCGACACCCCAGCCCCGACGGCAGCCGACAGCCCGG
TCATGGCGACTGACATTGATATGCTAATTGACCTCGGCCTGGACCTCTCCGACAGCGATCTGGACGAG
GACCCCCCCGAGCCGGCGGAGAGCCGCCGCGACGACCTGGAATCGGACAGCAACGGGGAGTGTTCCTC
GTCGGACGAGGACATGGAAGACCCCCACGGAGAGGACGGACCGGAGCCGATACTCGACGCCGCTCGCC
CGGCGGTCCGCCCGTCTCGTCCAGAAGACCCCGGCGTACCCAGCACCCAGACGCCTCGTCCGACGGAG
CGGCAGGGCCCCAACGATCCTCAACCAGCGCCCCACAGTGTGTGGTCGCGCCTCGGGGCCCGGCGACC
GTCTTGCTCCCCCGAGCGGCACGGGGGCAAGGTGGCCCGCCTCCAACCCCCACCGACCAAAGCCCAGC
CTGCCCGCGGCGGACGCCGTGGGCGTCGCAGGGGTCGGGGTCGCGGTGGTCCCGGGGCCGCCGATGGT
TTGTCGGACCCCCGCCGGCGTGCCCCCAGAACCAATCGCAACCCGGGGGGACCCCGCCCCGGGGGGGG
GTGGACGGACGGCCCCGGCGCCCCCCATGGCGAGGCGTGGCGCGGAAGTGAGCAGCCCGACCCACCCG
GAGGCCCGCGGACACGGAGCGTGCGCCAAGCACCCCCCCCGCTAATGACGCTGGCGATTGCCCCCCCG
CCCGCGGACCCCCGCGCCCCGGCCCCGGAGCGAAAGGCGCCCGCCGCCGACACCATCGACGCCACCAC
GCGGTTGGTCCTGCGCTCCATCTCCGAGCGCGCGGCGGTCGACCGCATCAGCGAGAGCTTCGGCCGCA
GCGCACAGGTCATGCACGACCCCTTTGGGGGGCAGCCGTTTCCCGCCGCGAATAGCCCCTGGGCCCCG
GTGCTGGCGGGCCAAGGAGGGCCCTTTGACGCCGAGACCAGACGGGTCTCCTGGGAAACCTTGGTCGC
CCACGGCCCGAGCCTCTATCGCACTTTTGCCGGCAATCCTCGGGCCGCATCGACCGCCAAGGCCATGC
GCGACTGCGTGCTGCGCCAAGAAAATTTCATCGAGGCGCTGGCCTCCGCCGACGAGACGCTGGCGTGG
TGCAAGATGTGCATCCACCACAACCTGCCGCTGCGCCCCCAGGACCCCATTATCGGGACGGCCGCGGC
GGTGCTGGATAACCTCGCCACGCGCCTGCGGCCCTTTCTCCAGTGCTACCTGAAGGCGCGAGGACTCT
GTGGACTCGATGAGCTCTGCTCTCGCCGACGGCTCGCCGATATCAAAGATATCGCCTCTTTTGTCTTC
GTGATCCTCGCTCGCTTGGCTAATAGAGTGGAAAGAGGAGTGGCCGAAATTGATTATGCCACATTGGG
CGTGGGCGTGGGCGAAAAAATGCACTTTTATTTACCTGGCGCTTGTATGGCTGGATTGATTGAGATTC
TGGATACCCATAGACAAGAATGCTCCTCTAGAGTGTGTGAACTGACCGCTTCCCATATTGTGGCTCCT
CCCTATGTCCATGGAAAGTACTTCTACTGTAATAGTCTCTTCTAG
(SEQ ID NO: 4)
GGATCCCAACGACCCCGCCCATGGGTCCCAATTGGCCGTCCCGTTACCAAGACCAACCCAGCCAGCGT
ATCCACCCCCGCCCGGGTCCCCGCGGAAGCGGAACGGTGTATGTGATATGCTAATTAAATACATGCCA
CGTACTTATGGTGTCTGATTGGTCCTTGTCTGTGCCGGAGGTGGGGCGGGGGCCCCGCCCGGGGGGCG
GAACTAGGAGGGGTTTGGGAGAGCCGGCCCCGGCACCACGGGTATAAGGACATCCACCACCCGGCCGG
TGGTGGTGTGCAGCCGTGTTCCAACCACGGTCACGCTTCGGTGCCTCTCCCCGATTCGGGCCCGGTCG
CTTGCTACCGGTGCGCCACCACCAGAGGCCATATCCGACACCCCAGCCCCGACGGCAGCCGACAGCCC
GGTCATGGCGACTGACATTGATATGCTAATTGACCTCGGCCTGGACCTCTCCGACAGCGATCTGGACG
AGGACCCCCCCGAGCCGGCGGAGAGCCGCCGCGACGACCTGGAATCGGACAGCAACGGGGAGTGTTCC
TCGTCGGACGAGGACATGGAAGACCCCCACGGAGAGGACGGACCGGAGCCGATACTCGACGCCGCTCG
CCCGGCGGTCCGCCCGTCTCGTCCAGAAGACCCCGGCGTACCCAGCACCCAGACGCCTCGTCCGACGG
AGCGGCAGGGCCCCAACGATCCTCAACCAGCGCCCCACAGTGTGTGGTCGCGCCTCGGGGCCCGGCGA
CCGTCTTGCTCCCCCGAGCGGCACGGGGGCAAGGTGGCCCGCCTCCAACCCCCACCGACCAAAGCCCA
GCCTGCCCGCGGCGGACGCCGTGGGCGTCGCAGGGGTCGGGGTCGCGGTGGTCCCGGGGCCGCCGATG
GTTTGTCGGACCCCCGCCGGCGTGCCCCCAGAACCAATCGCAACCCGGGGGGACCCCGCCCCGGGGCG
GGGTGGACGGACGGCCCCGGCGCCCCCCATGGCGAGGCGTGGCGCGGAAGTGAGCAGCCCGACCCACC
CGGAGGCCCGCGGACACGGAGCGTGCGCCAAGCACCCCCCCCGCTAATGACGCTGGCGATTGCCCCCC
CGCCCGCGGACCCCCGCGCCCCGGCCCCGGAGCGAAAGGCGCCCGCCGCCGACACCATCGACGCCACC
ACGCGGTTGGTCCTGCGCTCCATCTCCGAGCGCGCGGCGGTCGACCGCATCAGCGAGAGCTTCGGCCG
CAGCGCACAGGTCATGCACGACCCCTTTGGGGGGCAGCCGTTTCCCGCCGCGAATAGCCCCTGGGCCC
CGGTGCTGGCGGGCCAAGGAGGGCCCTTTGACGCCGAGACCAGACGGGTCTCCTGGGAAACCTTGGTC
GCCCACGGCCCGAGCCTCTATCGCACTTTTGCCGGCAATCCTCGGGCCGCATCGACCGCCAAGGCCAT
GCGCGACTGCGTGCTGCGCCAAGAAAATTTCATCGAGGCGCTGGCCTCCGCCGACGAGACGCTGGCGT
GGTGCAAGATGTGCATCCACCACAACCTGCCGCTGCGCCCCCAGGACCCCATTATCGGGACGGCCGCG
GCGGTGCTGGATAACCTCGCCACGCGCCTGCGGCCCTTTCTCCAGTGCTACCTGAAGGCGCGAGGACT
CTGTGGACTCGATGAGCTCTGCTCTCGCCGACGGCTCGCCGATATCAAAGATATCGCCTCTTTTGTCT
TCGTGATCCTCGCTCGCTTGGCTAATAGAGTGGAAAGAGGAGTGGCCGAAATTGATTATGCCACATTG
GGCGTGGGCGTGGGCGAAAAAATGCACTTTTATTTACCTGGCGCTTGTATGGCTGGATTGATTGAGAT
TCTGGATACCCATAGACAAGAATGCTCCTCTAGAGTGTGTGAACTGACCGCTTCCCATATTGTGGCTC
CTCCCTATGTCCATGGAAAGTACTTCTACTGTAATAGTCTCTTCTAGGCTCTCGACAATCAACCTCTG
GATTACAAAATTTGTGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTCCGGGACTTT
CGCTTTCCCCCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCTCAATCCAGC
GGACCTTCCTTCCCGCGGCCTGCTGCCGGCTCTGCGGCCTCTTCCGCGTCTCCGCCTTCGCCCTCAGA
CGAGTCGGATCTCTCTTTGGGCCGCCTCCCCGCCTGGTACCTTTAAGACCAATGACTTACAAGGCAGC
TGTAGATCTTAGCCACTTTTTAAAAGAAAAGGGGGGACCTGGTACCTTTAAGACCAATGACTTACAAG
GCAGCTGTAGATCTTAGCCACTTTTTAAAAGAAAAGGGGGGACTGGAAGGGCTAATTCACTCCCAACG
AAGATAAGATCTGCTTTTTGCTTGTACTGGGTCTCTCTGGTTAGACCAGATCTGAGCCTGGGAGCTCT
CTGGCTAACTAGGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTGTGGAAAATCTC
TAGCA

### Example 6 Generation of All-In-One (A-1-0) rHSV Vectors with Complete ICP27 Deletion and Insertion into the UL54 Locus with Both a Human Dystrophin Minigene and an AAV Rep/Cap Coding Sequence

This example provides another embodiment of the instant A-I-O platform wherein the gene of interest (GOI, or human dystrophin minigene in this case) and the AAV Rep/Cap coding sequence are both inserted into a different portion of the HSV genome—the ICP27 locus or UL54, instead of the UL43 locus in Example 1 or 2. The general procedure as outlined in Examples 1 and 2 can be followed to generate the A-I-O construct with the insertion within the ICP27/UL54 deletion, as can other methods known in the art. For example, here, one-step recombination using a bacterial selection marker was used to insert the Pro-AAV cassette and the RepCap cassette, instead of using galK-mediated insertion as used in Example 1 or 2.

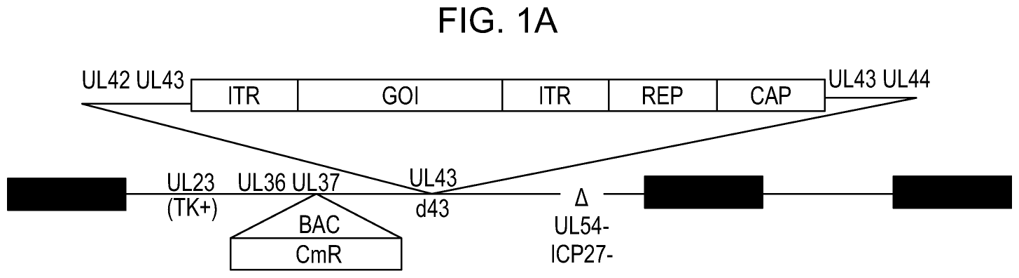

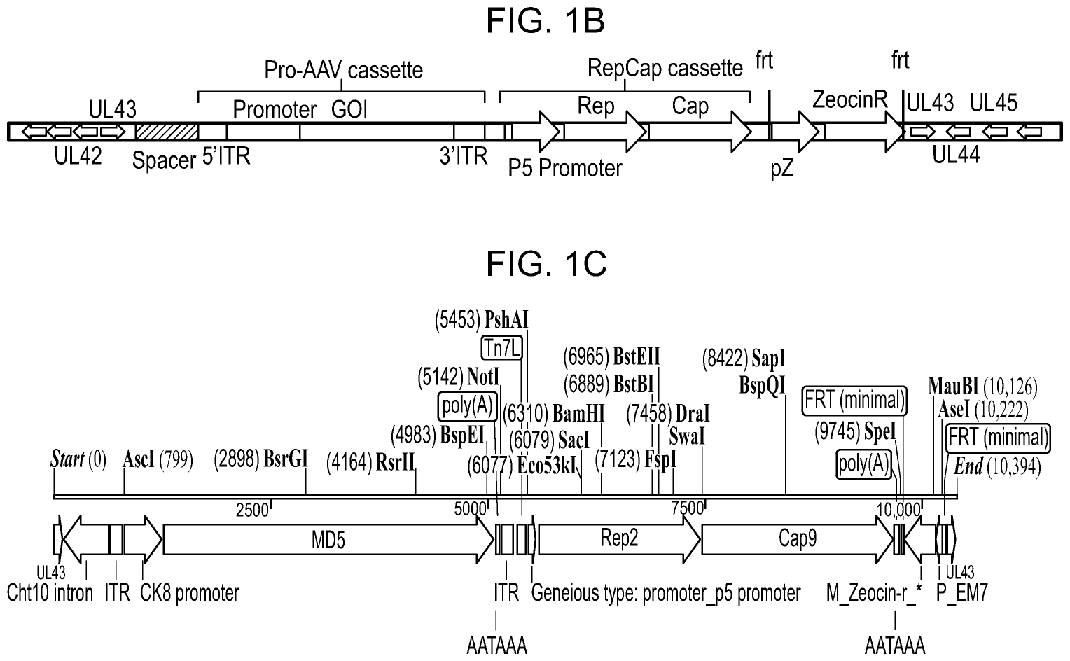

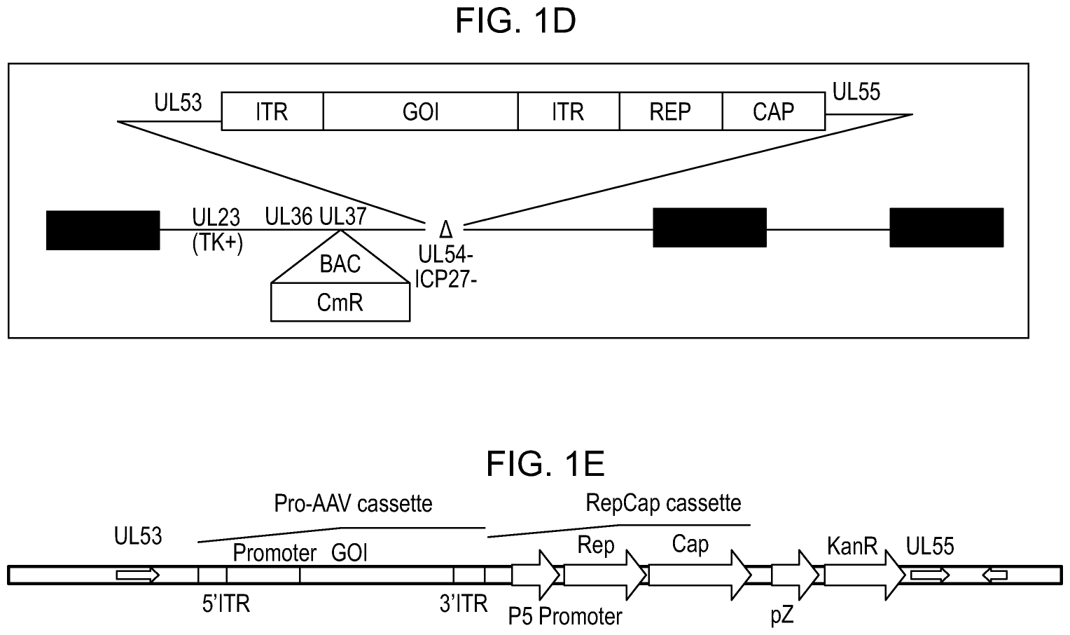

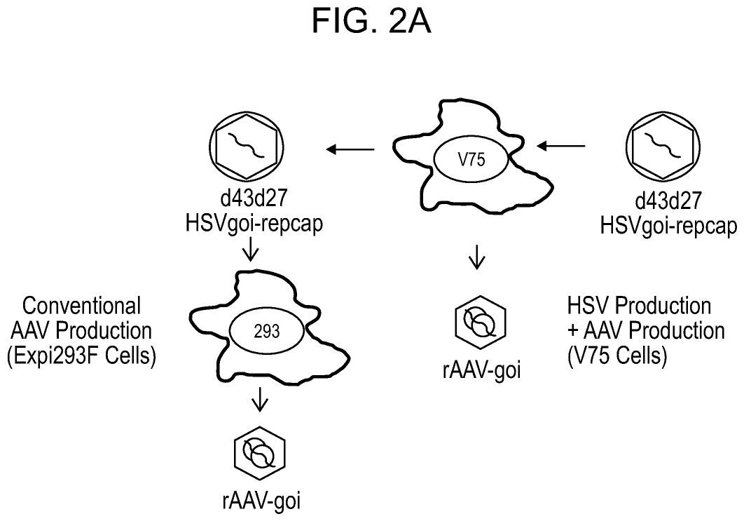

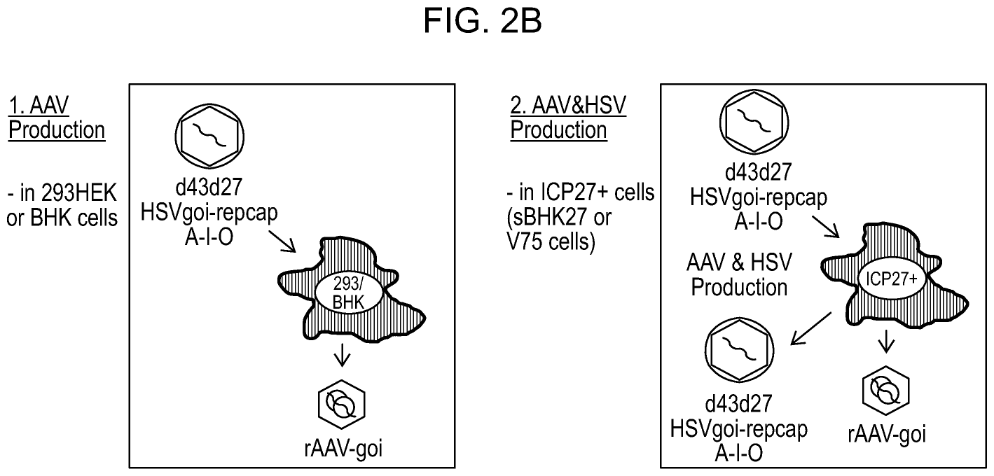

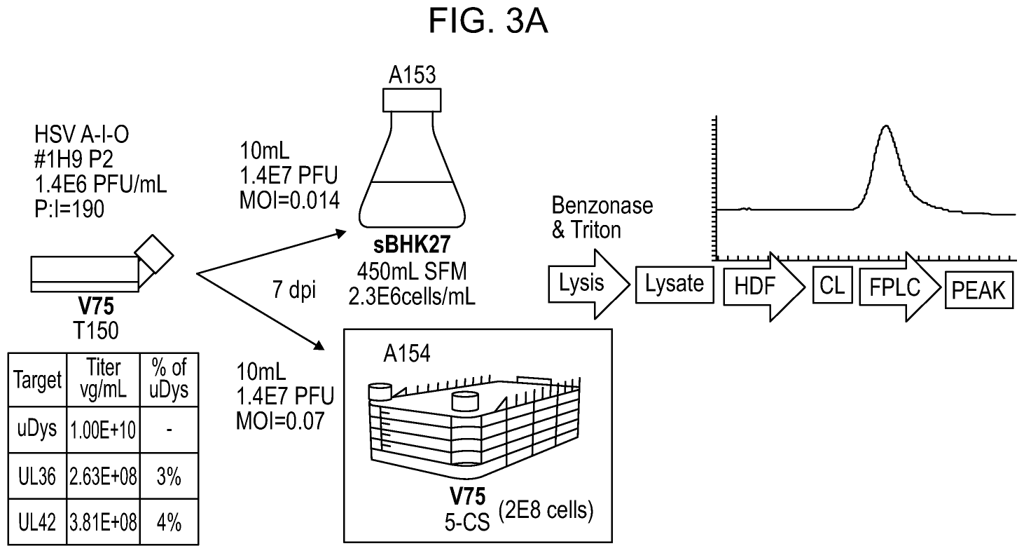

To produce this embodiment of the A-I-O HSV vectors, the ICP27/UL54 locus was completely deleted, followed by insertion of both a gene-of-interest (GOI) flanked by AAV ITR sequences, and an AAV Rep/Cap coding sequence into the same UL54 locus. See illustrative drawings in FIGS. 1D and 1E.

Specifically, ICP27 gene (UL54), including its promoter and coding sequence, was first completely deleted from the wild-type HSV-1 KOS 1.1 strain genome integrated in a bacterial artificial chromosome (BAC) vector (HSV-1 KOS 1.1-BAC), by using homologous recombination in electrocompetent E. coli. Independently isolated ICP27-deleted HSV-1-BAC clones with 2.1-kb (2.077 bp) deletion in UL54 gene were tested using the V27 and Vero cell plaque assay to show that robust production of ICP27-deleted HSV-1 virus can be achieved from these ICP27-deleted HSV-1-BAC clones with little to no rcHSV contamination (data not shown). SEQ ID NO: 1 is a polypeptide sequence of ICP27 HSV-1 strain KOS 1.1 ICP27 peptide (SEQ ID NO: 1; GenPept Accession AAF43147).

The remaining procedures of Example 1 or 2 were followed generally as described.

### Example 7 A-I-O UL54 HSV Vectors Minimized DNA Mis-packaging in AAV

Similarly to Example 4, this example demonstrates a surprising finding of greatly reduced AAV mis-packaging when the subject A-I-O HSV vectors were used to produce AAV in Expi293 cells. Here, GOI and RepCap were inserted at the UL54 locus.

It was found that, when the repcap cassette and the GOI cassette were both inserted into the UL54 locus, only at relatively low frequency (e.g., <1%), certain HSV vector sequences outside the ITR sequences are mis-packaged into the same AAV viral particles, resulting in mis-packaged AAV viral packages having non-productive HSV vector sequences.

As shown in FIG. 8, mis-packaging in the traditional two-HSV vector system happens at a rough frequency of about 8-20% compared to the correctly packaged GOI.

Similarly as shown in Example 4, when the subject A-I-O HSV vector (with both GOI and RepCap coding sequence inserted at the UL54 locus) was used to produce AAV directly in sBHK27 cells, the rate of AAV mis-packaging was significantly reduced (see Tables A-B).

Specifically, sBHK27 cells were infected by the subject A-I-O HSV vector having both GOI and RepCap coding sequence inserted in the UL54 locus with completely deleted ICP27 gene (AIO-U154 HSV), at MOI of either 0.02 or 0.1. Harvested AAV viral particles were assessed for the content of the packaged DNA via ddPCR using standard protocol (see Example 4). Primer pairs specific for named HSV genes were used for ddPCR to detect the presence of specific HSV sequences packaged in the AAV. The results are summarized in the tables below.

TABLE A
AAV/HSV Titers in Crude Lysate of
AIO Ul54 HSV-infected sBHK27 Cells

| sBHK27 | μDYS vg/ml | Ul53 vg/ml |
|---|---|---|
| MOI 0.02 | 1.81E+10 | 4.33E+07 |
| MOI 0.1 | 1.99E+10 | 9.92E+07 |

* vg/mL: vector genome/mL

TABLE B
Percent (%) Mispackaging of HSV Genes in AAV Viral Particles
% Mispackaging

| | % Ul53 |
|---|---|
| MOI 0.02 | 0.24% |
| MOI 0.1 | 0.50% |

It is apparent that, at either the high (0.1) or low (0.02) MOI, producer cell lines (i.e., sBHK27 cells in this case) infected by the subject AIO U154 HSV vector generated AAV viral stocks with extremely low percentage of mispackaged DNA—generally at or lower than 0.5%, as compared to the 8-20% range seen in conventional two-vector-based HSV systems.

### Example 8 Similar HSV Production Yields for Both the Traditional Two-HSV System and the A-I-O HSV System in Vero Cells

This example demonstrates A-I-O HSV vector of the present invention, unexpectedly and through a simple yet elegant approach, can reach similar HSV production yield as that of the traditional dual HSV vector system as demonstrated in the ICP27-expressing V27 HSV packaging cell line.

In this experiment, V27 cells grown in 5-cell stack (5-CS) tissue culture flasks were either infected by the traditional dual HSV system vectors (e.g., one HSV vector carrying the repcap structural genes, and another HSV vector carrying any gene of interest or GOI), or by the A-I-O HSV system the present invention (in which both the repcap structural genes and the GOI are in the same HSV vector inserted at the same locus). The MOI used for the infections were all about 0.015-0.02.

In this experiment, the two HSV vectors used for the dual HSV vector system were “RC9” and “001,” the former of which comprising the rep and cap genes for use in producing AAV9 viral particles, and the latter of which comprising the coding sequence for a microdystrophin (μD5). At 3.5 days post infection (3.5 DPI), HSV titers were determined based on ddPCR that amplified a marker gene on the HSV viral genome (e.g., UL36, in this case).

The results in the table below showed that both HSV vectors (RC9 and 001) produced similar titers of HSV viral particles, in the range of about 6E+08/mL:

| | |
|---|---|
| 5 cell stack | 3.5 DPI |
| V27 cells | HSV Titer ddPCR |

| HSV Vectors | 001 HSV | RC9 HSV |
|---|---|---|
| Post Harvest | 6.33E+08 | 5.85E+08 |

In a similar experiment, V27 cells grown in 5-cell stack tissue culture bottles were infected by A-I-O HSV vector of the present invention (with both repcap genes and GOI inserted at the UL43 locus), at an MOI of about 0.02. Again, at 3.5 DPI, HSV titer was determined based on ddPCR that amplified the same HSV marker gene, UL36, and showed that the AIO HSV vector of the present invention produced a titer of 5.69E+08/mL.

HSV titers were also measured at 7 DPI for AIO HSV vector of the present invention. It was found that, compared to the 3.5 DPI harvest, the HSV titers decreased from 5.69E+08/mL to 3.17E+08/mL.

| | 3.5 DPI | | 7 DPI | |
|---|---|---|---|---|
| 5 cell stack | AIO HSV | | AIO HSV | |
| V27 cells | HSV Titer | AAV Titer | HSV Titer | AAV Titer |
| Post Harvest | 5.69E+08 | 3.62E+09 | 3.17E+08 | 2.13E+10 |

Thus, the titers of the A-I-O HSV vectors of the present invention were comparable to that of the traditional dual HSV vector system.

A-I-O HSV vectors of the present invention can also produce AAV particles (while the traditional two-HSV system vectors would not, as each of the GOI HSV and rep/cap HSV vectors are separately produced). The above table also reports AAV titer results of the present invention at both 3.5 DPI and 7 DPI measured using a GOI (μD5) marker. Notably, unlike the HSV titer decrease, the AAV titers increased by about 4-6 fold from the 3.5 DPI and 7 DPI harvests. Therefore, another aspect of the present invention may include harvesting AAV at a later date than 3.5 DPI, such as 7 DPI, resulting in a multi-fold increase in AAV titer over the relatively stable HSV titer.

### Example 9 Similar AAV Production Yields with Dramatically Reduced Mis-Packaging for the A-1-O HSV System in Expi293 Cells as Compared to the Traditional Dual HSV System

This example demonstrates A-I-O HSV vector of the present invention with similar AAV production yield in Expi293 production cells, as compared to that of the traditional two-HSV (dual HSV) system, yet simultaneously having much lower rate of mis-packaging.

In this experiment, A-I-O HSV vector of the present invention had both the repcap and GOI (μD5 microdystrophin) inserted into the UL43 locus. Mis-packaging for the present invention was measured by determining the ratio of Ul42 titer (i.e., 4.95E+08) to AAV titer (i.e., 5.64E+10), or 0.88%.

AAV mis-packaging for the dual HSV system was determined using GOI HSV vector where the GOI was inserted into the UL23 locus. Mis-packaging was measured by determining the ratio of U124 titer (i.e., 2.57E+09) to AAV titer (i.e., 6.29E+10), or 4.09%, about a 465% increase over the mis-packaging rate of the A-I-O system of the present invention.

Both systems, however, had comparable AAV titers, in the range of 6E+10 (see table below). Thus the data again confirms the superiority of the A-I-O system over the traditional dual HSV system for AAV production.

| | HSV Type | AAV Titer | Ul24 Titer | Ul42 Titer | % mispack |
|---|---|---|---|---|---|
| EXPI | Dual HSV | 6.29E+10 | 2.57E+09 | — | 4.09% |
| 293 | AIO-HSV | 5.64E+10 | — | 4.95E+08 | 0.88% |

1. A recombinant replication-defective Herpes Simplex Virus (HSV) vector, comprising:
(1) a defective ICP27 gene that impairs or otherwise does not support replication of said HSV;
(2) a gene of interest (GOI) flanked by AAV ITR sequences, inserted at or within a non-essential locus (e.g., the UL43 locus) or a replaceable essential locus (e.g., the UL54 locus) of said HSV; and,
(3) an expression cassette comprising a coding sequence for AAV Rep and Cap proteins, inserted at or within the non-essential locus (e.g., the UL43 locus) or the replaceable essential locus (e.g., the UL54 locus) of the HSV.

2. The rHSV vector of claim 1, wherein said defective ICP27 gene comprises/is a complete deletion of the UL54/ICP27 locus of the HSV (e.g., a complete deletion of SEQ ID NO: 2, or a coding sequence for SEQ ID NO: 1).

3. The rHSV vector of claim 1 or 2, comprising an intact UL42 locus and an intact UL44 locus and/or an intact UL53 locus and an intact UL55 locus (optionally further comprising an intact UL23 locus and/or an intact US5 locus).

4. The rHSV vector of any one of claims 1-3, wherein said HSV is Herpes Simplex Virus-1 (HSV-1) or Herpes Simplex Virus-2 (HSV-2).

5. The rHSV vector of any one of claims 1-4, wherein said HSV is a strain of HSV-1, such as KOS, KOS 1.1, KOS 1.1A, KOS-37, KOS63, KOS79, McKrae, Stain 17, F17, or McIntyre.

6. The rHSV vector of any one of claims 1-5, wherein said GOI is a functional equivalent of dystrophin (e.g., a dystrophin minigene encoding a functional micro-dystrophin protein).

7. The rHSV vector of any one of claims 1-5, wherein said GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

8. The rHSV vector of any one of claims 1-5, wherein the GOI is a microdystrophin gene.

9. The rHSV vector of claim 8, wherein the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

10. The rHSV vector of claim 9, wherein the microdystrophin gene comprises a coding sequence for R16 and R17 spectrin-like repeats for the full length dystrophin protein (such as one described in U.S. Pat. No. 7,892,824).

11. The rHSV vector of claim 10, wherein the microdystrophin gene comprises a coding sequence for the R1, R16, R17, R23, and R24 spectrin-like repeats of the full-length dystrophin protein (such as the microdystrophin gene described in PCT/US2016/013733).

12. The rHSV vector of any one of claims 1-11, wherein said AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, or AAV-DJ.

13. The rHSV vector of claim 12, wherein the tropism of the AAV include skeletal muscle (such as AAV1, AAV6, AAV7, AAV8, or AAV9, preferably AAV9).

14. The rHSV vector of any one of claims 1-13, wherein said AAV ITR, said AAV Rep, and said AAV Cap are from the same or different AAVs.

15. The rHSV vector of claim 14, wherein said AAV ITR is AAV2 ITR, said AAV Rep is Rep2 from AAV2, and said AAV Cap is Cap9 from AAV9.

16. The rHSV vector of any one of claims 1-15, wherein said coding sequence for AAV Rep and Cap proteins is under the transcriptional control of a promoter, such as an AAV p5 promoter, an upstream HSV promoter, a modified p5 promoter lacking RBE (Rep-Binding Element), or a ubiquitous promoter (such as CMV promoter, EF1a promoter, CAG promoter, CB promoter etc).

17. The rHSV vector of any one of claims 1-16, further comprising a removable transcription unit inserted at or within the UL43 locus of the HSV, wherein the removable transcription unit comprises a selection marker (such as a Kanamycin or zeocin resistance gene Kan^{R} or Zeo^{R}) under the transcriptional control of a selection marker promoter (such as a Zeo^{R} promoter).

18. The rHSV vector of any one of claims 1-16, further comprising a removable transcription unit inserted at or within the UL54 locus of the HSV, wherein the removable transcription unit comprises a selection marker (such as a Kanamycin or zeocin resistance gene Kan^{R} or Zeo^{R}) under the transcriptional control of a selection marker promoter (such as a Zeo^{R} promoter).

19. The rHSV vector of claim 17 or 18, wherein the removable transcription unit is flanked by a pair of flippase recognition target (FRT) sites.

20. A recombinant replication-defective Herpes Simplex Virus (HSV) viral particle, comprising the rHSV vector of any one of claims 1-19.

21. A host cell comprising the recombinant rHSV vector of any one of claims 1-19.

22. The host cell of claim 21, which does not comprise a coding sequence for ICP27, and which optionally further comprises helper virus proteins required for AAV packaging.

23. The host cell of claim 22, which is a HEK293 cell (such as an Expi293F cell), a HeLa cell, an A549 cell, a BHK cell, or an insect cell (such as Sf9).

24. The host cell of claim 21, which comprises a coding sequence for, and is capable of expressing ICP27 or a functional equivalent thereof.

25. The host cell of claim 24, comprising:
(1) a coding sequence for said ICP27 or said functional equivalent thereof, operatively linked to a promoter capable of directing the transcription of said coding sequence in the host cell;
(2) a polyadenylation site 3′ to the coding sequence; and,
(3) optionally, one or more multi-cloning site(s).

26. The host cell of claim 24 or 25, wherein said ICP27 has the amino acid sequence of SEQ ID NO: 1, or is at least 60%, 70%, 75%, 80%, 85%, 90%, 95%, 97%, 98%, 99%, 99.2%, 99.4%, 99.6%, or 99.8% identical to SEQ ID NO: 1.

27. The host cell of claim 25 or 26, wherein the promoter comprises nucleotides 1-538 of SEQ ID NO: 2, nucleotides 127-538 of SEQ ID NO: 2, nucleotides 113,139-113,550 of GenBank Accession No. KT887224, or nucleotides 113,013-113,550 of GenBank Accession No. KT887224.

28. The host cell of any one of claims 24-27, wherein said coding sequence is partially or fully codon-optimized for expression in a mammalian host cell.

29. The host cell of claim 28, wherein the most 3′ 300-350 nucleotides of the coding sequence are codon-optimized for expression in the mammalian host cell.

30. The host cell of any one of claims 24-29, wherein said polyadenylation site is a bovine growth hormone (bGH) polyadenylation site.

31. The host cell of any one of claims 23-30, wherein the coding sequence for said ICP27 comprises a mutation that reduces inhibition of host cell pre-mRNA splicing, while permitting HSV late gene expression.

32. The host cell of claim 31, wherein the mutation is vBS3.3 double mutation, vBS4.3 double mutation, or vBS5.3 double mutation.

33. The host cell of claim 31 or 32, which is a Vero cell (such as a V75 cell) or a BHK cell (such as a sBHK27 cell).

34. A method of simultaneously propagating/amplifying/producing the recombinant replication-defective HSV viral particle of claim 20, and a recombinant adeno-associated virus (AAV) viral particle encapsidating the GOI flanked by said AAV ITR, the method comprising: infecting the host cell of any one of claims 24-33 with the recombinant replication-defective HSV viral particle of claim 20.

35. The method of claim 34, further comprising harvesting the recombinant replication-defective HSV of claim 20 from the infected host cell of any one of claims 24-33.

36. A method of producing a recombinant Adeno-Associated Virus (rAAV) comprising a gene of interest (GOI) coding sequence flanked by AAV ITR sequences, said method comprising infecting a production host cell of any one of claims 21-33, with the recombinant replication-defective HSV viral particle of claim 20.

37. The method of claim 36, wherein the production cell line is BHK, Vero, or HEK293.


SEQUENCE LISTING (4 sequences)

SEQ ID NO: 1 - protein, 512 aa, Human alphaherpesvirus 1
MATDIDMLIDLGLDLSDSDLDEDPPEPAESRRDDLESDSNGECSSSDEDMEDPHGEDGPEPILDAARPAVRPSRPEDPGVPSTQTPRPTERQGPNDPQPAPHSVWSRLGARRPSCSPERHGGKVARLQPPPTKAQPARGGRRGRRRGRGRGGPGAADGLSDPRRRAPRTNRNPGGPRPGAGWTDGPGAPHGEAWRGSEQPDPPGGPRTRSVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTAAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 2 - DNA, 2077 bp, Human alphaherpesvirus 1
cgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 3 - DNA, 1881 bp, Artificial Sequence
  Description of Artificial Sequence Synthetic polynucleotide
ccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctag

SEQ ID NO: 4 - DNA, 2929 bp, Artificial Sequence
  Description of Artificial Sequence Synthetic polynucleotide
ggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggactctgtggactcgatgagctctgctctcgccgacggctcgccgatatcaaagatatcgcctcttttgtcttcgtgatcctcgctcgcttggctaatagagtggaaagaggagtggccgaaattgattatgccacattgggcgtgggcgtgggcgaaaaaatgcacttttatttacctggcgcttgtatggctggattgattgagattctggatacccatagacaagaatgctcctctagagtgtgtgaactgaccgcttcccatattgtggctcctccctatgtccatggaaagtacttctactgtaatagtctcttctaggctctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctccgccttcgccctcagacgagtcggatctctctttgggccgcctccccgcctggtacctttaagaccaatgacttacaaggcagctgtagatcttagccactttttaaaagaaaaggggggacctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca